(19) United States
(12) Patent Application Publication
(10) Pub. No.: US 2024/0366540 A1
(43) Pub. Date: Nov. 7, 2024

(54) MITOFUSIN INHIBITORS AND USES THEREOF

(71) Applicant: Albert Einstein College of Medicine, Bronx, NY (US)

(72) Inventors: Evripidis Gavathiotis, Roslyn, NY (US); Emmanouil Zacharioudakis, Bronx, NY (US)

(21) Appl. No.: 18/736,027

(22) Filed: Jun. 6, 2024

Related U.S. Application Data
(63) Continuation-in-part of application No. PCT/US22/80900, filed on Dec. 5, 2022.
(60) Provisional application No. 63/286,264, filed on Dec. 6, 2021.

(51) Int. Cl. A61K 31/167; A61K 31/137; A61K 31/196; C07C 215/50; C07C 233/88; C07C 235/56

(52) U.S. Cl. CPC A61K 31/167; A61K 31/137; A61K 31/196; C07C 215/50; C07C 233/88; C07C 235/56

(57) ABSTRACT

Disclosed herein are mitofusin inhibitors which are capable of inducing mitochondrial fission, decreasing mitochondrial respiration, TCA metabolism, and inducing mitochondrial outer membrane permeabilization that leads to caspase activation and DNA damage signaling. Also disclosed are methods of treating diseases or conditions associated with imbalanced mitochondrial dynamics.

### CROSS-REFERENCE TO RELATED APPLICATIONS

This patent document is a continuation-in-part application of PCT/US22/80900, filed Dec. 5, 2022, which claims the benefit of U.S. Provisional Patent Application No. 63/286,264, filed on Dec. 6, 2016, and U.S. Provisional Patent Application No. 63/366,746, filed on Jun. 21, 2022, the contents of which are herein incorporated by reference into the subject application.

### REFERENCE TO AN ELECTRONIC SEQUENCE LISTING

This application contains a Sequence Listing which has been submitted electronically in xml format and is hereby incorporated by reference in its entirety. Said xml copy, created on Jul. 12, 2024, is named SeqList-182219-00219.xml and is 18,726 bytes in size.

### TECHNICAL FIELD

Disclosed herein are mitofusin inhibitors and their use in treating various diseases and conditions.

### BACKGROUND

Mitochondria provide eukaryotic cells with a means to generate significant quantities of ATP through oxidative phosphorylation. They are also potentially dangerous organelles because of their abilities to generate reactive oxygen and nitrogen species and mediate cell death. In part because of these liabilities, mitochondria engage in repeated cycles of fission and fusion. These events, referred to in the aggregate as mitochondrial dynamics, are tightly coupled with the elimination of damaged mitochondria through macroautophagy (mitophagy) and with mitochondrial biogenesis, processes which together maintain the health of the mitochondrial collective in a cell.

Mitochondrial fusion is a two step process; the first step requires the fusion of the outer mitochondrial membrane (OMM), which is mediated by mitofusin-1 (MFN1) and mitofusin-2 (MFN2). MFN1 and MFN2 are mediator of mitochondrial fusion. They are mitochondrial membrane proteins that interact with each other to facilitate mitochondrial targeting. The second step requires the fusion of the inner mitochondrial membrane, which is mediated by optic atrophy-1 (OPA1). Loss of either MFN1/2 or OPA1 proteins results in a network of hyper-fragmented mitochondria. While both MFN1 and MFN2 deficient cells display a clearly fragmented mitochondrial network, MFN1 knockout cells display severe mitochondrial fragmentation with formation of small spheres of similar size and MFN2 knockout cells display mitochondrial spheres or ovals of variable but larger size. Overexpression of either MFN1 or MFN2 in wild type cells leads to extensive mitochondrial clustering in the perinuclear area. However, overexpression of MFN1 in MFN2 KO and MFN2 in MFN1 KO cells restores mitochondrial fusion, highlighting the degree of redundancy between the MFN proteins.

Multiple diseases ranging from cancer, heart disease, diabetes, and neurodegenerative disorders, as well as aging, are accompanied by disruption of mitochondrial dynamics. Often this is characterized by an imbalance in mitochondrial fission over fusion.

Thus, there is a need to develop new agents for treating diseases or conditions associated with imbalance in mitochondrial dynamics.

### SUMMARY

Mitofusins reside in the outer mitochondrial membrane and regulate mitochondrial fusion, a physiological process that impacts diverse cellular processes. Mitofusins are activated by conformational changes and subsequently oligomerize to enable mitochondrial fusion. Mitofusin activation increases mitochondrial fusion and functionality, whereas mitofusin inhibition decreases mitochondrial fusion and functionality. Remarkably, mitofusin inhibition also induces minority mitochondrial outer membrane permeabilization (MOMP), followed by sub-lethal caspase-3/7 activation, which induces DNA damage and upregulated DNA damage response genes. This patent disclosure provides compounds that induce fragmented mitochondria and lead to decreased membrane potential, mitochondrial respiration and ATP production.

An aspect of the patent document provides a compound of Formula I and pharmaceutically acceptable salts thereof. The compound is generally represented as follows:

Ar¹-L-X—Ar² Formula I
wherein
Ar¹ is a 6-membered aryl or 6-membered heteroaryl, wherein the aryl or heteroaryl is substituted with one more substituents selected from the group consisting of deuterium, OC₁₋₆alkyl, SC₁₋₆alkyl, CN, OH, SH, COOH, halogen, NO₂, N(R^{m})₂, C(O)OR^{m}), C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl) C₁₋₆alkyl, OC(O)N(R^{m})₂, C(O)SR^{m}, OC₁₋₆alkyleneOC₁₋₆alkyl, OC₁₋₆alkylencO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, provided that at least one of the one more substituents is a first otho substituent positioned ortho to L and is a hydrogen-bond donor;
Ar² is a 6-10 membered aryl or 5-10 membered heteroaryl, wherein the aryl or heteroaryl is substituted with one more substituents selected from the group consisting of deuterium, OC₁₋₆alkyl, SC₁₋₆alkyl, CN, OH, SH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₆alkyl, dihydroxyC₁₋₁₀alkyl, C₃₋₆cycloalkyl, C(═NC₁₋₆alkyl) C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, provided that at least one of the one more substituents is a second othro substituent positioned ortho to X and is selected from the group consisting of OC₁₋₆alkyl, SC₁₋₆alkyl, C₁₋₄alkyl, CN, halogen, C₁₋₆alkylene-CN, OC₁₋₆alkylene-CN, haloC₁₋₆alkyl, SC₁₋₆alkylene-CN, C₂₋₆alkynyl, C₂₋₆alkenyl, C₁₋₆alkylSO₂ (sulfone);
L is C₁₋₃alkylene optionally substituted with an oxo (═O);
X is C₁₋₃alkylene or NR″;
R^{m} each is independently hydrogen or C₁₋₆alkyl or halo-C₁₋₆alkyl; and
Rⁿ is hydrogen, C₁₋₆alkyl, halo-C₁₋₆alkyl, or C(O)C₁₋₆alkyl.

Also provided are pharmaceutical compositions comprising a therapeutically effective amount of a compound of Formula (I) disclosed herein or a pharmaceutically acceptable salt thereof, and a pharmaceutically acceptable excipient.

Another aspect provides a method for treating a disease or condition. The method includes administering to a subject in need thereof a compound of formula (I), a pharmaceutically acceptable salt thereof, or a pharmaceutical composition thereof.

Another aspect provides a method for inhibiting mitofusin-mediated mitochondrial fusion. The method includes contacting a cell with an effective amount of the compound of formula (I) or a pharmaceutically acceptable salt thereof or a pharmaceutical composition thereof.

### DESCRIPTION OF THE DRAWINGS

FIG. 1A shows a pharmacophore hypothesis based on the sidechains of the HR1-amino acids: Val372, Met376, His380 interacting with HR2 comprising of 3 hydrophobic points, one aromatic ring and one hydrogen bond donor.

FIG. 1B shows the chemical structure of MASM7.

FIG. 2A shows a pharmacophore hypothesis based on the sidechains of the HR1-amino acids: Leu408, Ala412, Tyr415 interacting with HR2 comprising 2 hydrophobic points, one aromatic ring and one hydrogen bond donor or acceptor.

FIG. 2B shows the chemical structures of MFI8 and its analogs.

FIG. 2C illustrates a pharmacophore model of an inhibitor of mitofusin-mediated mitochondrial fusion.

FIG. 3 shows that MFI8 reduces viability in leukemia cells. MFI8 concentration responsively reduces viability in MV-411 (A) and MOLM13 (B) cells. Cell titer Glo was used to measure viability. Cells were treated with MFI8 for 72 hr (n=3, mean±SD).

FIG. 4 shows that MFI8 reduces viability in melanoma cells. MFI8 concentration responsively reduces viability in A375 (A) and SKML-30 (B) cells. Cell titer Glo was used to measure viability. Cells were treated with MFI8 for 72 hr (n=3, mean±SD).

FIG. 5 shows that MFI8 reduces viability in pancreatic cancer cells. MFI8 concentration responsively reduces viability in BXPC3 cells. Cell titer Glo was used to measure viability. Cells were treated with MFI8 for 72 hr (n=3, mean±SD).

FIG. 6 shows that MFI8 reduces viability in colon cancer cells. MFI8 concentration responsively reduces viability in H508 (A) and HT29 (B) cells. Cell titer Glo was used to measure viability. Cells were treated with MFI8 for 72 hr (n=3, mean±SD).

FIG. 7 shows that MFI8 reduces viability in lung cancer cells. MFI8 concentration responsively reduces viability in Calu-6 (A), H2405 (B) cells. Cell titer Glo was used to measure viability. Cells were treated with MFI8 for 72 hr (n=3, mean±SD).

FIG. 8 shows that MFI8 induces apoptosis in Calu-6 cells. Calu-6 cells were treated with MFI8 (20 μM). Cell death was monitored at the indicated timepoints using Annexin V as a readout. Data represent mean (n=3).

### DETAILED DESCRIPTION

This patent document discloses mitofusin inhibitors and their effects in inducing mitochondrial fission, decreasing mitochondrial respiration, TCA metabolism, and inducing mitochondrial outer membrane permeabilization that leads to caspase activation and DNA damage signaling. Multiple diseases including for example cancer, heart disease, diabetes, and neurodegenerative disorders, as well as aging, are accompanied by disruption of mitochondrial dynamics. Accordingly, the compounds disclosed herein possess great therapeutic potential for treating diseases associated with imbalance in mitochondrial dynamics.

While the following text may reference or exemplify specific embodiments of a compound, substituent, or use thereof, it is not intended to limit the scope of the compound, substituent or its use to such particular references or examples. Various modifications may be made by those skilled in the art, in view of scientific and practical considerations, such as replacement of a substituent or treatment of other diseases.

The articles “a” and “an” as used herein refer to “one or more” or “at least one,” unless otherwise indicated. That is, reference to any element or component of an embodiment by the indefinite article “a” or “an” does not exclude the possibility that more than one element or component is present.

The term “acyl” refers to —C(O)CH₃, —C(O)CH₂CH₃, —C(O)CH₂CH₂CH₃, or-C(O)CH₂CH₂CH₂CH₃.

The term “alkyl” refers to a hydrocarbon or a hydrocarbon chain which may be either straight-chained or branched. The term “C₁₋₆ alkyl” refers to alkyl groups having 1, 2, 3, 4, 5 or 6 carbon atoms. Non-limiting examples include groups such as CH₃, (CH₂)₂CH₃, CH₂CH(CH₃) CH₃, and the like. Similarly, the term “C₂₋₅ alkyl” refers to alkyl groups having 2, 3, 4 or 5 carbon atoms.

The term “alkylene” refers to a divalent hydrocarbon or a hydrocarbon chain which may be either straight-chained or branched. Non-limiting examples include groups such as CH₂, (CH₂)₂CH₂, CH₂CH(CH₃) CH₂, and the like. A C₁₋₃alkylene includes alkylenes with 1, 2 or 3 carbons such as CH₂, (CH₂)₂, CHCH₃, (CH₂)₃, and CH(CH₃) CH₂.

The term “cycloalkyl” refers to saturated and partially unsaturated cyclic hydrocarbon groups having 3 to 12 ring carbons, for example 3 to 8 carbons, and as a further example 3 to 6 carbons, wherein the cycloalkyl group additionally is optionally substituted. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “aryl” group refers to a C₆₋₁₄ aromatic moiety comprising one to three aromatic rings, which is optionally substituted. Examples of aryl groups include, without limitation, phenyl, naphthyl, anthracenyl, fluorenyl, and dihydrobenzofuranyl.

The term “alkenyl” refers to a carbon chain containing a carbon-carbon double bond moiety. Non-limiting examples of alkenyl groups include ethylenyl, 1-propenyl, allyl and 2-butenyl.

The term “alkynyl” group refers to a carbon chain containing a carbon-carbon triple bond moiety. Non-limiting examples of alkynyl groups include ethynyl, 1-propanyl, propargyl and 2-butynyl.

The term “haloalkyl” refers to a C₆₋₁₀alkyl chain, straight or branched, in which one or more hydrogen has been replaced by a halogen. Non-limiting examples of haloalkyls include CHF₂, CFH₂, CF₃, CH₂CHF₂, CH₂CH₂Cl, CH₂CF₃, and CH₂CH₂F. In some embodiments, the alkyl in haloalkyl has 1, 2, 3 or 4 carbons.

The term “heteroalkyl” refers to a C₆₋₁₀alkyl group, straight or branched, wherein one or more carbon atoms in the chain are replaced by one or more heteroatoms selected from the group consisting of O, S, N and NR^{m}. In some embodiments, the alkyl in heteroalkyl has 1 to 10 carbons. In some embodiments, the alkyl in heteroalkyl has 2, 3, 4 or more than 2 carbons.

The term “hydroxyalkyl” refers to a C₆₋₁₀alkyl chain, straight or branched, wherein a carbon is substituted with a hydroxyl group. The carbon the hydroxyl is attached to is a primary carbon or secondary carbon. In some embodiments, the alkyl in hydroxylalkyl has 2, 3, 4 or more than 2 carbons.

The term “dihydroxyalkyl” refers to a C₂₋₁₀alkyl chain, straight or branched, wherein two carbons are each substituted with a hydroxyl group. In some embodiments, the alkyl in dihydroxylalkyl has 2, 3, 4 or more than 2 carbons.

The term “heterocyclyl” or “heterocyclic” group is a ring structure having from about 3 to about 12 atoms, for example 4 to 8 atoms, wherein one or more atoms are selected from the group consisting of N, O, and S, the remainder of the ring atoms being carbon. The heterocyclyl may be a monocyclic, a bicyclic, a spirocyclic or a bridged ring system. Examples of heterocyclic groups include, without limitation, epoxy, azetidinyl, aziridinyl, azocanyl, azepanyl, diazepanyl, dihydrofuranyl, tetrahydrofuranyl, tetrahydropyranyl, oxazepanyl, pyrrolidinyl, pyrrolidinonyl, piperidinyl, pipcrazinyl, imidazolidinyl, thiazolidinyl, thiooxazepanyl, dithianyl, trithianyl, dioxolanyl, oxazolidinyl, oxazolidinonyl, decahydroquinolinyl, piperidonyl, 4-piperidinonyl, thiomorpholinyl, thiomorpholinyl 1,1 dioxide, morpholinyl, oxazepanyl, azabicyclohexanes, azabicycloheptanes and oxa azabiocycloheptanes. Specifically excluded from the scope of this term are compounds having adjacent annular O and/or S atoms.

The term “heteroaryl” refers to groups having 5 to 14 ring atoms, preferably 5, 6, 9, or 10 ring atoms; having 6, 10, or 14 π electrons shared in a cyclic array; and having, in addition to carbon atoms, from one to three heteroatoms per ring selected from the group consisting of N, O, and S. Examples of heteroaryl groups include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

The term “halogen” refers to F, Cl, Br or I.

The term “subject” refers to humans or animals including for example sheep, horses, cattle, pigs, dogs, cats, rats, mice, birds, and reptiles. Preferably, the subject is a human or other mammal.

The term “effective amount” or “therapeutically effective amount” of a compound is an amount that is sufficient to ameliorate, or in some manner reduce a symptom or stop or reverse progression of a condition, or negatively modulate or inhibit activity. Such amount may be administered as a single dosage or may be administered according to a regimen, whereby it is effective.

The term “hydrogen bond donor” refers to a group containing a hydrogen, which can be form a hydrogen bond with another electronegative atom such as F, N or O. Non-limiting examples of hydrogen bond donor include OH and NH2, which can share its hydrogen with electron rich atoms to form a hydrogen bond.

The term “hydrogen bond acceptor” refers to a group or atom rich in electrons, which can form a hydrogen bond with a hydrogen bond donor. Non-limiting examples of hydrogen bond acceptor include O, N and F.

The term “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

The term “pharmaceutically acceptable carrier” refers to a chemical compound that facilitates the delivery or incorporation of a compound or therapeutic agent into cells or tissues.

The term “pharmaceutically acceptable salts” means salts of compounds of the present invention which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tannic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, ethylenediamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth cds., Verlag Helvetica Chimica Acta, 2002).

The term “pharmaceutical composition” refers to a mixture of a compound disclosed herein with other chemical components, such as diluents or additional carriers. The pharmaceutical composition facilitates administration of the compound to an organism. Multiple techniques of administering a pharmaceutical composition exist in the art including, but not limited to, oral, injection, aerosol, parenteral, intranasal, sublingual, inhalational, and topical administration. In some embodiments, pharmaceutically acceptable salts of the compounds disclosed herein are provided.

The term “treating” or “treatment” of any disease or condition refers, in some embodiments, to ameliorating the disease or disorder (i.e., arresting or reducing the development of the disease or at least one of the clinical signs and symptoms thereof). In some embodiments “treating” or “treatment” refers to ameliorating at least one physical parameter, which may not be discernible by the subject. In some embodiments, “treating” or “treatment” refers to modulating the disease or disorder, either physically, (e.g., stabilization of a discernible symptom), physiologically, (e.g., stabilization of a physical parameter), or both. In some embodiments, “treating” or “treatment” refers to delaying the onset of the disease or disorder, or even preventing the same. “Prophylactic treatment” is to be construed as any mode of treatment that is used to prevent progression of the disease or is used for precautionary purpose for persons at risk of developing the condition.

Mitochondria fuse, divide and interact with other organelle structures to regulate cellular fitness and fate while they produce the majority of energy to sustain cellular activity. They are highly dynamic organelles constantly undergoing the physiological process of fusion and fission which regulates mitochondrial morphology and dynamics. Among different cell types or within the same type of cells, mitochondria morphology varies among small spheres, short rods or long tubules. This dynamism allows mitochondria to exchange components (e.g., lipid membranes, proteins), promote repair and removal of defective mitochondria, thus maintaining mitochondrial function and quality. Furthermore, mitochondrial fusion and fission enable mitochondria to meet cellular energy demands in response to environmental stimuli. Fused mitochondria often lead to increased oxidative phosphorylation and mitochondrial membrane potential. In contrast, fragmented mitochondria often correlate with reduced function, decreased mitochondrial membrane potential and oxidative phosphorylation.

### Compounds

An aspect of the disclosure provides a compound of formula I or a pharmaceutically acceptable salt thereof, which are capable of regulating directly MFN1/2 activity and subsequently mitochondrial fusion. The compound binds directly to the recombinant HR2 domain of MFN2 and in cells to intact protein, decreases the GTP-dependent MFN2 higher-order oligomers, and therefore impedes mitochondrial fusion by directly interfering with the tethering permissive structure of MFNs.

Ar¹-L-X—Ar² Formula I
wherein:
Ar¹ is a 6-membered aryl or 6-membered heteroaryl, wherein the aryl or heteroaryl is substituted with one more substituents selected from the group consisting of deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₆alkyl, dihydroxyC₁₋₁₀alkyl, C₃₋₆cycloalkyl, C(═NC₁₋₆alkyl) C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, provided that at least one of the one more substituents is positioned ortho to L and is a hydrogen-bond donor;
Ar² is a 6-10 membered aryl or 5-10 membered heteroaryl, wherein the aryl or heteroaryl is substituted with one more substituents selected from the group consisting of deuterium, OC₁₋₆alkyl, SC₁₋₆alkyl, CN, OH, SH, halogen, NO₂, N(R^{m})₂, C(O)OR^{m}), C(O)N(R^{m}1)₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl) C₁₋₆alkyl, OC(O)N(R^{m})₂, C(O)SR^{m}, OC₁₋₆alkyleneOC₁₋₆alkyl, OC₁₋₆alkylencO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkylencO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, provided that at least one of the one more substituents is positioned ortho to X and is a hydrophobic group or selected from the group of OC₁₋₆alkyl, SC₁₋₆alkyl, C₁₋₄alkyl, CN, halogen, C₁₋₆alkylene-CN, OC₁₋₆alkylene-CN, haloC₁₋₆alkyl, SC₁₋₆alkylene-CN, C₂₋₆alkynyl, C₂₋₆alkenyl, and C₁₋₆alkylSO₂ (sulfone);
L is C₁₋₃alkylene optionally substituted with an oxo (═O);
X is C₁₋₃alkylene or NRⁿ;
R^{m} each is independently hydrogen or C₁₋₆alkyl or halo-C₁₋₆alkyl; and
Rⁿ is hydrogen, C₁₋₆alkyl, halo-C₁₋₆alkyl, C(O)C₁₋₆alkyl.

In some embodiments, Ar¹ is a substituted phenyl

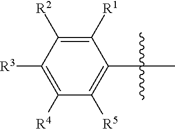

wherein the first ortho substituent is R¹ selected from the group consisting of OH, SH, COOH, N(R^{m})₂, C(O)N(R^{m})₂, hydroxyC₁₋₆alkyl, dihydroxyC₁₋₁₀alkyl, C₃₋₆cycloalkyl, NR^{m}SO₂C₁₋₆alkyl, and S(O)OH, wherein at least one R^{m} in N(R^{m})₂, C(O)N(R^{m})₂, and NR^{m}SO₂C₁₋₆alkyl is hydrogen;
R², R³, R⁴, and R⁵ are independently selected from the group consisting of hydrogen, deuterium, OC₁₋₆alkyl, SC₁₋₆alkyl, CN, OH, SH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl) C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂. R^{m} and R^{m} are as described above.

In some embodiments, Ar¹ is a substituted phenyl comprising a substituent, ortho to L, selected from the group consisting of OH, SH, COOH, N(R^{m})₂, C(O)N(R^{m})₂, hydroxyC₁₋₆alkyl, dihydroxyC₁₋₁₀alkyl, C₃₋₆cycloalkyl, NR^{m}SO₂C₁₋₆alkyl, or S(O)OH, wherein at least one R^{m} in N(R^{m})₂, C(O)N(R^{m})₂, and NR^{m}SO₂C₁₋₆alkyl is hydrogen. In some embodiments, Ar¹ is a substituted phenyl comprising an OH ortho to L.

In some embodiments, Ar¹ is a substituted phenyl comprising a substituent ortho to L and one or more additional substituents selected from the group consisting of CN, halogen, NO₂, C(O)OR^{m}, C(O)N(R^{m})₂, C(O)C₁₋₆alkyl, haloC₁₆alkyl, haloC₁₋₆alkyleneO, C₁₋₆alkyl, C(O)SR^{m}, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂.

In some embodiments, Ar¹ further comprises a substituent, para to the first ortho substituent, selected from the group consisting of CN, halogen, NO₂, C(O)OR^{m}, C(O)N(R^{m}))₂, C(O)C₁₋₆alkyl, haloC₁₋₆alkyl, haloC₁₋₆alkyleneO, C₁₋₆alkyl, C(O)SR^{m}, C₂₋₆alkynyl, C₂₋₆alkenyl, SO₂N(R^{m}1)₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂. In some embodiments, the para substituent is a F, C₁ or Br. In some embodiments, the ortho substituent is OH and the substituent para to it is C₁.

In some embodiments, Ar² is a substituted phenyl, pyridinyl, or pyrimidinyl. In some embodiments, Ar² comprises an ortho substituent selected from OC₁₋₆alkyl, SC₁₋₆alkyl, C₁₋₄alkyl, CN, halogen, C₁₋₆alkylene-CN, OC₁₋₆alkylene-CN, haloC₁₋₆alkyl, SC₁₋₆alkylene-CN, C₂₋₆alkynyl, C₂₋₆alkenyl, and C₁₋₆alkylSO₂ (sulfone).

In some embodiments, Ar² is a substituted phenyl

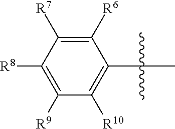

Wherein the second ortho substituent is R⁶,
R⁷, R⁸, R⁹, and R¹⁰ are independently selected from the group consisting of hydrogen, deuterium, OC₁₋₆alkyl, SC₁₋₆alkyl, CN, OH, SH, halogen, NO₂, N(R^{m})₂, C(O)OR^{m}), C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl) C₁₋₆alkyl, OC(O)N(R^{m})₂, C(O)SR^{m}, OC₁₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, provided that at least one of the one more substituents is a second ortho substituent positioned ortho to X and is selected from the group consisting of OC₁₋₆alkyl, SC₁₋₆alkyl, CN, halogen, C₁₋₆alkylene-CN, OC₁₋₆alkylene-CN, haloC₁₋₆alkyl, SC₁₋₆alkylene-CN, C₂₋₆alkynyl, C₂₋₆alkenyl, and C₁₋₆alkylSO₂ (sulfone). R^{m} and R^{m} are as described above.

In some embodiments, the ortho substituent of Ar² is selected from the group consisting of OC₁₋₆alkyl, SC₁₋₆alkyl, C₁₋₄alkyl, CN, halogen, haloC₁₋₆alkyl, C₂₋₆alkynyl, and C₂₋₆alkenyl. In some embodiments, ortho substituent is C₁₋₄alkyl.

In some embodiments, Ar² comprises a meta substituent next to the second ortho substituent, wherein the meta substituent is selected from the group consisting of C₁₋₄alkyl, OC₁₋₆alkyl, SC₁₋₆alkyl, CN, halogen, C₁₋₆alkylene-CN, OC₁₋₆alkylene-CN, haloC₁₋₆alkyl, SC₁₋₆alkylene-CN, C₂₋₆alkynyl, C₂₋₆alkenyl, C₁₋₆alkylSO₂ (sulfone). In some embodiments, Ar² is phenyl, the second ortho substituent is C₁₋₄alkyl, the meta substituent is C₁₋₄alkyl. In some embodiments, Ar² further comprises a second meta substituent (meta to X and para to the second ortho substituent) selected from SC₁₋₆alkyl, CN, OH, SH, halogen, NO₂, N(R^{m})₂, and C(O)OR^{m}, C(O)N(R^{m})₂. In some embodiments, Ar² is phenyl, the second ortho substituent is C₁₄alkyl, the meta substituent is C₁₄alkyl, and the substituent para to the second ortho substituent is selected from halogen, C(O)OR^{m} (e.g. COOH), C(O)N(R^{m})₂, hydroxyC₁₋₆alkyl, dihydroxyC₁₋₁₀alkyl, C₃₋₆cycloalkyl, NR^{m}SO₂C₁₋₆alkyl, and S(O)OH. The scope of R^{m} and Rⁿ are as described above.

In some embodiments, L is C₁alkylene optionally substituted with an oxo (═O). In some embodiments, L is methylene, ethylene, or CH(CH₃). In some embodiments, L is C(O).

In some embodiments, X is NH. In some embodiments, X is NC₁₋₆alkyl. In some embodiments, X is NC(O)C₁₋₆alkyl.

In some embodiments, the compound is Formula II, wherein L is methylene, CH(CH₃), SO₂, or C(O), X is NH or NC(O)C₁₋₆alkyl, R⁴ is halogen, and R¹ is OH.

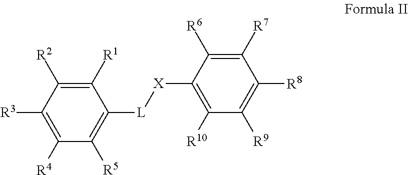

In some embodiments of Formula II, R⁶ and R⁷ are independently selected from the group consisting of hydrogen, OC₁₋₆alkyl, C₁₄alkyl, and halogen, and wherein at least one of R⁶ and R⁷ is not hydrogen. In some embodiments of Formula II, R⁶ and R⁷ are independently C₁₋₄alkyl or halogen.

In some embodiments of Formula II, any one, two, or three of R⁸, R⁹, and R¹⁰ is a halogen. In some embodiments of Formula II, only one of R⁸, R⁹, and R¹⁰ is a halogen. In some embodiments, the halogen is F, Cl or Br.

Nonlimiting examples of Formula II include the following:

| | L | X | R¹ | R² | R³ | R⁴ | R⁵ | R⁶ | R⁷ | R⁸ | R⁹ | R¹⁰ |
|---|---|---|---|---|---|---|---|---|---|---|---|---|
| 1 | C(O) | NH | OH | Me | Me | Cl | Me | H | H | H | H | H |
| 2 | C(O) | NH | OH | Me | Me | Cl | Me | Me | Me | F | H | H |
| 3 | C(O) | NC(O)Me | OH | Me | Me | Cl | Me | Me | H | Cl | H | H |
| 3 | C(O) | NH | OH | Me | Me | Cl | Me | H | Me | Me | H | Me |
| 5 | C(O) | NH | OH | Me | Me | F | Me | F | H | Me | H | Me |
| 6 | CHCH₃ | NH | OH | Me | Me | Cl | Me | H | Cl | H | H | H |
| 7 | CHCH₃ | NH | OH | Me | Me | Cl | Me | F | Me | F | H | F |
| 8 | CHCH₃ | NH | OH | H | H | Cl | H | Et | Cl | H | H | H |
| 9 | C(O) | NH | OH | Me | Me | Cl | Me | OMe | H | F | H | F |
| 10 | C(O) | NH | OH | Me | Me | Cl | Me | F | Cl | Et | H | Et |
| 11 | C(O) | NH | OH | Me | Me | Cl | Me | H | H | OMe | H | OMe |
| 12 | C(O) | NH | OH | Me | Me | Cl | Me | Me | H | Me | F | Me |
| 13 | C(O) | NH | OH | Me | Me | Cl | Me | Me | Me | Me | Me | Me |
| 14 | C(O) | NH | OH | Me | Me | Cl | Me | H | Me | H | H | H |
| 15 | C(O) | NH | OH | Me | Me | Cl | Me | F | H | F | H | F |
| 16 | C(O) | NH | OH | Me | Me | Cl | Me | H | F | H | H | H |
| 17 | C(O) | NH | OH | Me | Me | Cl | Me | F | H | F | H | F |
| 18 | C(O) | NH | OH | Me | Me | Cl | Me | Et | F | Et | H | Et |
| 19 | C(O) | NH | OH | Me | Me | Cl | Me | OMe | Et | OMe | H | OMe |
| 20 | C(O) | NH | OH | Me | Me | Cl | Me | F | OMe | Me | H | Me |
| 21 | C(O) | NH | OH | Me | Me | Cl | Me | H | F | Me | H | Me |
| 22 | CHCH₃ | NH | OH | H | H | Cl | H | Me | H | H | H | H |
| 23 | SO₂ | NH | OH | H | H | Cl | H | Me | Me | F | Et | F |
| 24 | SO₂ | NH | OH | Et | Et | Cl | Et | H | Me | H | Et | H |
| 25 | C(O) | NH | OH | Et | Et | F | Et | F | H | F | Me | F |
| 26 | C(O) | NH | OH | H | H | Cl | H | H | F | Et | Me | Et |
| 27 | C(O) | NH | OH | H | H | Cl | H | F | H | OMe | H | OMe |
| 28 | CHCH₃ | NH | OH | Et | Et | Cl | Et | Et | F | Me | F | Me |
| 29 | SO₂ | NH | OH | H | H | Cl | H | H | Et | Me | H | Me |
| 30 | SO₂ | NH | OH | H | H | Cl | H | H | OMe | H | F | H |
| 31 | C(O) | NH | OH | H | H | Cl | H | H | F | F | Et | F |
| 32 | C(O) | NH | OH | H | H | F | H | Me | H | H | OMe | H |
| 33 | C(O) | NH | OH | H | H | Cl | H | Me | Me | F | Me | F |
| 34 | C(O) | NH | OH | H | H | Cl | H | H | Me | Et | Me | Et |
| 35 | C(O) | NH | OH | Me | Me | Cl | Me | F | H | OMe | H | OMe |
| 36 | C(O) | NH | OH | Me | Me | Cl | Me | H | F | Me | F | Me |
| 37 | C(O) | NH | OH | Me | Me | Cl | Me | F | H | Me | H | Me |
| 38 | C(O) | NH | OH | Me | Me | Cl | Me | Et | F | H | F | H |
| 39 | C(O) | NH | OH | Me | Me | Cl | Me | OMe | Et | F | Et | F |
| 40 | C(O) | NH | OH | Me | Me | Cl | Me | F | OMe | H | OMe | H |
| 41 | C(O) | NH | OH | OMe | OMe | H | OMe | H | F | F | Me | F |
| 42 | C(O) | NH | OH | OMe | OMe | H | OMe | Me | H | Et | Me | Et |

In some embodiments, the compound is one of the following:

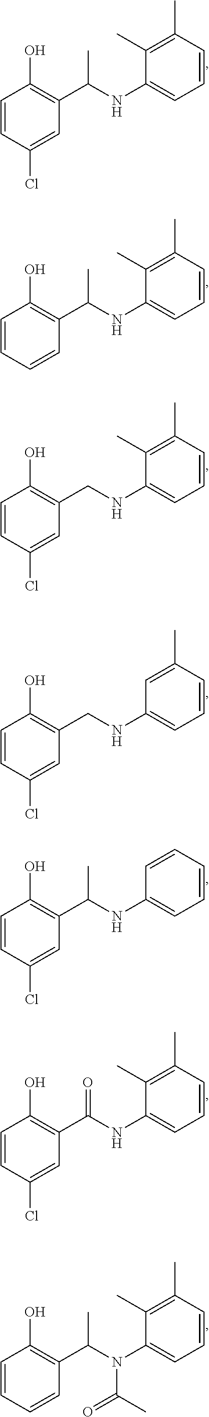

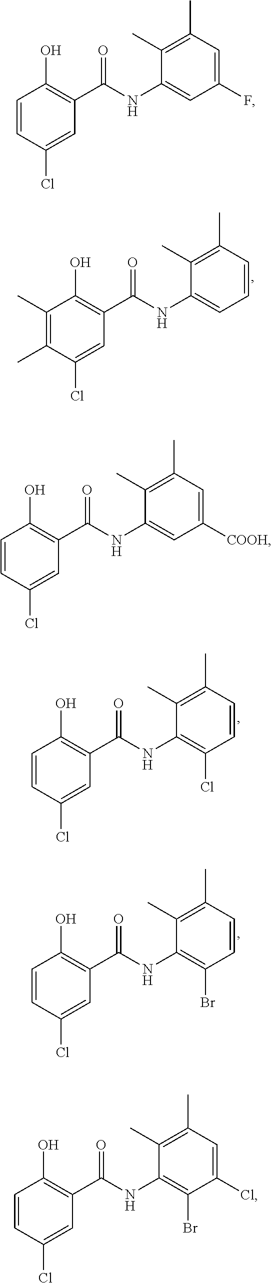 [SMILES: Cc1cc(F)cc(NC(=O)c2cc(Cl)ccc2O)c1C ; Cc1cccc(NC(=O)c2cc(Cl)c(C)c(C)c2O)c1C ; Cc1cc(C(=O)O)cc(NC(=O)c2cc(Cl)ccc2O)c1C ; Cc1ccc(Cl)c(NC(=O)c2cc(Cl)ccc2O)c1C ; Cc1ccc(Br)c(NC(=O)c2cc(Cl)ccc2O)c1C ; Cc1cc(Cl)c(Br)c(NC(=O)c2cc(Cl)ccc2O)c1C]

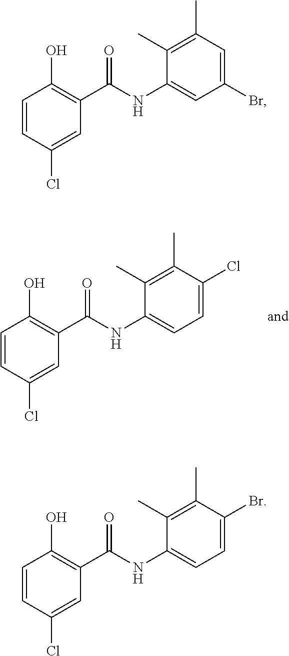 [SMILES: Cc1cc(Br)cc(NC(=O)c2cc(Cl)ccc2O)c1C ; Cc1c(Cl)ccc(NC(=O)c2cc(Cl)ccc2O)c1C ; Cc1c(Br)ccc(NC(=O)c2cc(Cl)ccc2O)c1C]

### Pharmaceutical Composition and Kit

Another aspect of the patent specification provides a pharmaceutical composition comprising a compound of Formula I or a pharmaceutically acceptable salt thereof disclosed herein and a pharmaceutically acceptable carrier, excipient, or diluent. Compounds described in this patent specification may be formulated by any method well known in the art and may be prepared for administration by any route, including, without limitation, parenteral, peroral, sublingual, buccal, intrathecal, transdermal, topical, subcutaneous, intramuscular, intraperitoneal, intranasal, intratracheal, or intrarectal.

Nonlimiting examples of pharmaceutically acceptable carriers include physiologically acceptable surface active agents, glidants, plasticizers, diluents, excipients, smoothing agents, suspension agents, complexing agents, film forming substances, and coating assistants. Preservatives, stabilizers, dyes, sweeteners, fragrances, flavoring agents, and the like may be provided in the pharmaceutical composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents. Suitable exemplary binders include crystalline cellulose, sucrose, D-mannitol, dextrin, hydroxypropylcellulose, hydroxypropylmethylcellulose, polyvinylpyrrolidone, and the like. Suitable exemplary disintegrants include starch, carboxymethylcellulose, calcium carboxymethylcellulose, croscarmellose sodium, sodium carboxymethylstarch, and the like. Suitable exemplary solvents or dispersion media include water, alcohol (for example, ethanol), polyols (for example, glycerol, propylene glycol, and polyethylene glycol, sesame oil, corn oil, and the like), and suitable mixtures thereof that are physiologically compatible. Suitable exemplary solubilizing agents include polyethylene glycol, propylene glycol, D-mannitol, benzylbenzoate, cyclodextrins, ethanol, trisaminomethane, cholesterol, triethanolamine, sodium carbonate, sodium citrate, and the like. Suitable exemplary suspending agents include surfactants such as stearyltriethanolamine, sodium laurylsulfate, laurylaminopropionic acid, lecithin, benzalkonium chloride, benzethonium chloride, glycerin monostearate, coconut oil, olive oil, sesame oil, peanut oil, soya and the like; and hydrophilic polymers such as polyvinyl alcohol, polyvinylpyrrolidonc, sodium carboxymethylcellulose, methylcellulose, hydroxymethylcellulose, hydroxyethylcellulose, hydroxypropylcellulose, and the like. Suitable exemplary isotonic agent includes sodium chloride, glycerin, D-mannose, and the like. Suitable exemplary buffer agents include buffer solutions of salts, such as phosphate, acetates, carbonates, and citrates. Suitable exemplary soothing agents include benzyl alcohol, and the like. Suitable exemplary antiseptic substances include para-oxybenzoic acid esters, benzethonium chloride, benzalkonium chloride, chlorobutanol, benzyl alcohol, phenethyl alcohol, dehydroacetic acid, sorbic acid, and the like. Suitable exemplary antioxidants include sulfite salts, ascorbic acid, and the like. Suitable exemplary sealers include, but are not limited to HPMC (or hypromellose), HPC, PEG and combinations thereof. Suitable exemplary lubricants include magnesium stearate, calcium stearate, talc, colloidal silica, hardened oil and the like.

In further exemplary embodiments for solid preparations, carriers or excipients include diluents, lubricants, binders, and disintegrants. In exemplary embodiments for liquid preparations, carriers include solvents, solubilizing agents, suspending agents, isotonic agents, buffer agents, soothing agents, and the like. Acceptable additional carriers or diluents for therapeutic use and the general procedures for the preparation of pharmaceutical compositions are well known in the pharmaceutical art, and are described, for example, in Remington's Pharmaceutical Sciences, 18th Ed., Mack Publishing Co., Easton, PA (1990), which is incorporated herein by reference in its entirety.

The compound of Formula I may also be in a pharmaceutically acceptable salt form. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt, wherein the counterion includes, for example, chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

A related aspect provides a kit, which includes a compound of Formula I or a pharmaceutically acceptable salt thereof, or a pharmaceutical composition thereof and an instruction for treating or preventing certain diseases or conditions. In some embodiments, the kit further includes an additional secondary therapeutic agent.

In some embodiments, the secondary agent is an anti-cancer agent. In any of the methods or kit described herein, the administration or inclusion of a secondary agent having a cytotoxic effect on a cancer cell is contemplated. A cytotoxic effect refers to the depletion, elimination and/or the killing of target cells (i.e., tumor cells). The cytotoxic agent may be at least one selected from the group consisting of an antimetabolite, a mitotic inhibitor, an alkylating agent, an antibody-based EGFR inhibitor, an antibody based HER2/3 inhibitor, an angiogenesis inhibitor, a mTOR inhibitor, a CDK4 and CDK6 inhibitor or an aromatase inhibitor. The combination may include at least two cytotoxic agents. For example, the combination may include at least 2, at least 3, or at least 4 selected from the group consisting of an antimetabolite, a mitotic inhibitor, an alkylating agent, an angiogenesis inhibitor, or all of them.

The antimetabolite may be a drug that inhibits DNA synthesis in cells by suppressing formation of purines or pyrimidines, which are bases of a nucleotide. In one embodiment, the antimetabolite may be selected from the group consisting of Capecitabine, 5-Fluorouracil, Gemcitabine, Pemetrexed, Methotrexate, 6-Mercaptopurine, Cladribine, Cytarabine, Doxifludine, Floxuridine, Fludarabine, Hydroxycarbamide, decarbazine, hydroxyurea, and asparaginase. In a more specific embodiment, the antimetabolite is a base analog, with the term base analogs herein including nucleotide and nucleoside analogs in addition to purine base analogs such as 5-fluorouracil.

The mitotic inhibitor may be a microtubule-destabilizing agent, a microtubule-stabilizing agent, or a combination thereof. The mitotic inhibitor may be selected from taxanes, vinca alkaloids, cpothilone, or a combination thereof. In a specific embodiment, the mitotic inhibitor is a taxane, for example including but not limited to, paclitaxel, docetaxel and cabazitaxel. In another specific embodiment, the mitotic inhibitor is a vinca alkaloid or its derivative, for example including but not limited to, vinblastine, vincristine, vinflunine, vinorelbine, vincaminol, vinburnine, vineridine and vindesinc.

The mitotic inhibitor may be selected from BT-062, HMN-214, cribulin mesylate, vindesine, EC-1069, EC-1456, EC-531, vintafolide, 2-methoxyestradiol, GTx-230, trastuzumab emtansinc (T-DM1), crolibulin, D1302A-maytansinoid conjugates IMGN-529, lorvotuzumab mertansinc, SAR-3419, SAR-566658, IMP-03138, topotecan/vincristine combinations, BPH-8, fosbretabulin tromethamine, estramustine phosphate sodium, vincristine, vinflunine, vinorelbine, RX-21101, cabazitaxel, STA-9584, vinblastine, epothilone A, patupilone, ixabepilonc, Epothilone D, paclitaxel, docetaxel, DJ-927, discodermolide, cleutherobin, and pharmaceutically acceptable salts thereof or combinations thereof.

Non-limiting examples of checkpoint inhibitors include those that target PD-1, PD-L1, CTLA4 and TIGIT (T cell immunoglobulin and ITIM domain). Further examples include Ipilimumab (Yervoy®; blocking a checkpoint protein called CTLA-4); pembrolizumab (Keytruda®), Cemiplimab (Libtayo) and nivolumab (Opdivo®) (targeting another checkpoint protein called PD-1); atezolizumab (Tecentriq®), Avelumab (Bavencio), and Durvalumab (Imfinzi) (targeting PD-L1); MK-7684, Etigilimab/OMP-313 M32, Tiragolumab/MTIG7192A/RG-6058, BMS-986207, AB-154 and ASP-8374 (targeting TIGIT), and V-domain Ig suppressor of T cell activation (VISTA).

The EGFR inhibitors may be selected from erlotinib, gefitinib, lapatinib, canetinib, pelitinib, neratinib, (R,E)-N-(7-chloro-1-(1-(4-(dimethylamino) but-2-enoyl) azepan-3-yl)-1H-benzo[d]imidazol-2-yl)-2-methylisonicotinamide, Trastuzumab, Margetuximab, panitumumab, matuzumab, necitumumab, pertuzumab, nimotuzumab, zalutumumab, cetuximab, icotinib, afatinib, and pharmaceutically acceptable salt thereof. In one embodiment the EGFR inhibitor may be an antibody based EGFR inhibitor such as cetuximab and in another embodiment, it is necitumumab and yet in another embodiment it is pantitumumab. The molecularly targeted agent may be an anti-EGFR family antibody or a complex including the anti-EGFR family antibody. The anti-EGFR family antibody may be an anti-HER1 antibody, an anti-HER2 antibody, or an anti-HER4 antibody.

Further examples of agents for chemotherapy include SHP2 inhibitors (e.g. RMC-4550 and RMC-4630), phosphatase inhibitors (e.g. Tautomycin), CDK 4/6 inhibitors (abemaciclib (Lilly), palbociclib (Pfizer)), protein-protein interaction disruptors (BI 1701963), HSP90 inhibitor, tubulin inhibitor, apoptosis inhibitor, chemopreventative agent, and therapies targeting PBK/AKT/mTOR pathway.

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. Another example is Trastuzumab duocarmazine. There are currently more than 30 ADC drug candidates in various stages of clinical trials for cancer treatment. As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

### Method of Treating Diseases

Another aspect of the patent specification provides for methods for treating a disease or condition associated with imbalance between mitochondrial fission and fusion, including for example abnormal or elevated mitochondrial fusion and abnormal or decreased mitochondrial fission. The method includes administering to a subject in need thereof the compound of formula (I), a pharmaceutically acceptable salt thereof, or a corresponding pharmaceutical composition disclosed herein.

The compounds of this patent specification directly target mitofusins and impact on mitochondrial dynamics. Diseases or conditions treatable with the compounds and their pharmaceutically acceptable salts include for example neurodegenerative diseases, metabolic disease, cardiovascular diseases, autoimmune disease, hypertension, inflammatory disease, ageing and cancer.

In some embodiments, the disease treatable with the methods disclosed herein is cancer including for example breast cancer, colorectal cancer, gastric cancer, glioma, anal cancer, appendix cancer, bile duct cancer (i.e., cholangiocarcinoma), bladder cancer, brain tumor, cervical cancer, esophageal cancer, eye cancer, fallopian tube cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, gastric cancer, testicular cancer, throat cancer, thyroid cancer, uterine cancer, vaginal cancer, vulvar cancer, leukemia, lymphoma or a solid tumor, acute lymphoblastic leukemia (ALL), acute myeloid leukemia (AML), chronic lymphocytic leukemia (CLL) or chronic myeloid leukemia (CML), non-Hodgkin's lymphoma, myeloma, multiple myeloma, prostate cancer, skin cancer, colon cancer, brain cancer, head-neck cancer, glioma, glioblastoma, stomach cancer, cancer of the gastrointestinal tract, non-small cell lung cancer (NSCLC), small-cell lung carcinoma, adrenal carcinoma, renal cell carcinoma, soft-tissue sarcoma, rhabdomyosarcoma, Wilms' tumor.

Although inhibition of mitochondrial fusion induces minority MOMP, sub-lethal activation of caspases-3/7 and DNA damage as downstream events of minority MOMP, it does not induce cell death. Such insight provides a new opportunity to use inhibition of MFN1/2 activity to sensitize cells to other agents promoting apoptosis. As demonstrated in the example section, BV6, a SMAC mimetic that operates downstream of MOMP, when combined with MFI8 can synergistically induces cell death. Similar combinatorial strategies can have broad applications in cancer therapy by enhancing the activity of pro-apoptotic drugs to induce caspase-mediated apoptosis or evade resistance mechanisms. Furthermore, inhibition of mitochondrial fusion may alter the transcription of several genes that are involved in DNA repair and respiration, suggesting that changes in mitochondrial shape can impact gene transcription of diverse gene sets. These are possible homeostatic mechanisms that cells use to counteract mitochondrial stress.

In some embodiments, the disease treatable with the methods disclosed herein is a cardiovascular disease including for example arrhythmia, ischemic heart disease, hypertensive heart disease and pulmonary hypertensive heart disease, valvular disease, congenital heart disease, cardiomyopathy, stroke ischemic heart disease, cardiac ischemia-reperfusion injury, myocardial infarction, chemotherapy-induced cardiotoxicity, arteriosclerosis, heart failure, heart transplantation.

In some embodiments, the disease treatable with the methods disclosed herein is a metabolic disorder including for example type II diabetes, obesity, insulin resistance, sarcopenia, diabetes, acute liver failure, NASH, hepatosteatosis, alcoholic fatty liver, renal failure and chronic kidney disease.

In some embodiments, the disease treatable with the methods disclosed herein is a neurodegenerative diseases including for example Alzheimer's disease, Lewy body dementia, frontotemporal dementia, traumatic brain injury, prion diseases, Huntington's disease, Parkinson's disease, chronic traumatic encephalopathy, amyotrophic lateral sclerosis, mixed dementias, vascular dementia, hydrocephalus, and amyotrophic lateral sclerosis.

Also disclosed in this patent document is the use of a therapeutically effective amount of a compound of Formula I or a pharmaceutically acceptable salt thereof to treat a disease or condition. This patent document further provides a therapeutically effective amount of a compound of Formula I or a pharmaceutically acceptable salt thereof for use in the treatment of a disease or condition. The disease or condition, the means of administration, the dosage form and formulation, and the additional agents are the same as in the methods described herein.

Another aspect of the patent document discloses a method of inhibiting mitofusin 1 and/or mitofusin 2. The method is applicable to attenuating or inhibiting mitofusin-mediated mitochondrial fusion. The method includes contacting a cell containing mitofusin 1 and/or mitofusin 2 with an effective amount of the compound of formula (I) or the pharmaceutically acceptable salt thereof or pharmaceutical composition disclosed herein to inhibit mitofusin 1 and/or mitofusin 2. The compounds are also effective for promoting decreased mitochondrial respiration and functionality, decreased metabolites of TCA cycle and/or promoting mitochondrial outer membrane permeabilization that leads to sublethal caspase activation and DNA damage. In some embodiments, the contacting takes place in vitro. In some embodiments, the contacting takes place in vivo. Similarly, this patent document further provides an effective amount of a compound of Formula I or a pharmaceutically acceptable salt thereof for use in inhibiting mitofusin-mediated mitochondrial fusion. Also disclosed in this patent document is the use of an effective amount of a compound of Formula I or a pharmaceutically acceptable salt thereof to inhibit mitofusin-mediated mitochondrial fusion.

A related aspect provides a method of inducing or promoting apoptosis/cell death in tumor cells. The method includes contacting a cell with an effective amount of the compound of formula (I) or the pharmaceutically acceptable salt thereof or pharmaceutical composition disclosed herein. In some embodiments, the contacting takes place in vitro, or the compound or its salt is administered to a subject in need thereof. The scope of cancer is as descried above. In some embodiments, the cancer is leukemia, melanoma, pancreatic cancer, colon cancer, lung cancer, head and neck cancer, lymphoma, ovarian cancer, prostate cancer, breast cancer, kindcy cancer, liver cancer, or bladder cancer. As illustrated in the examples, a compound of Formula I or its salt inhibits MFN1/2 and/or fragmentation of mitochondria and is able to induce a robust apoptosis induction and cell death.

In some embodiments of any methods or uses disclosed herein, there includes the administration of an additional agent including for example SMAC mimetics, BH3 mimetics and other pro-apoptotic anti-cancer drugs. BH3 mimetics is a class of small molecules that antagonizes with the pro-apoptotic BH3 domains in the binding to the hydrophobic pocket of the anti-apoptotic BCL-2 family of proteins such as Bcl-2, Bcl-xL, Mcl-1. BH3 mimetics are essentially selective inhibitors of Bcl-2 or Bcl-xL or Mcl-1 proteins or inhibit more than one anti-apoptotic BCL-2 family of proteins and activate the intrinsic pathway of apoptosis by inducing mitochondrial outer membrane permeabilization (MOMP). BH3 mimetics have been used as an anti-cancer treatment in the clinic in various solid tumors and hematological malignancies as they induce cell death/apoptosis in cancer cells. SMAC mimetics is a class of small molecules that mimics the interaction of second mitochondria-derived activator of caspases (SMAC) with the inhibitor of apoptosis proteins (IAPs). SMAC mimetics are antagonists of cIAP1, cIAP2, XIAP proteins. Inhibition of IAPs by SMAC mimetics induces cell death/apoptosis in cancer cells by activating the intrinsic and/or the extrinsic pathway of apoptosis. SMAC mimetics are evaluated in the clinic as anti-cancer treatment against solid tumors and hematological malignancies.

Nonlimiting examples of SMAC mimetics include Birinapant (TL32711), GDC-0152, Xevinapant (AT406), Tolinapant (ASTX660), AZD5582, BV-6, SM-164, LCL161, and APG-1387. Nonlimiting examples of BH3 mimetics include Venetoclax (ABT-199), Lisaftoclax (APG-2575), S55746, DT2216, Navitoclax (ABT-263), ABT-737, APG-1252, A-1331852, A-115546, S64315 (MIK665), S63845, AMG-176 and AZD5991. When used in combination, the SMAC mimetic or BH3 mimetic can be administered prior to, simultaneously with, or subsequent to the administration of the compound of Formula I or a salt thereof. In some embodiments, the compound of Formula I or a salt thereof in the combination is in an effective amount to decrease cell viability by about 10%, about 20%, about 30%, about 40%, about 50%, about 60%, about 80%, or about 100% in comparison with the SMAC mimetic or BH3 mimetic administered by itself. In some embodiments, the compound of Formula I or a salt thereof in the combination is in an effective amount to increase caspase 3/7 activation by about 10%, about 20%, about 30%, about 40%, about 50%, about 60%, about 80%, or about 100% in comparison with the SMAC mimetic or BH3 mimetic administered by itself.

Another aspect of the patent document provides a method of sensitizing cells to caspase activation or to apoptosis/cell death. The method includes contacting a cell with an effective amount of the compound of formula (I) or the pharmaceutically acceptable salt thereof or pharmaceutical composition disclosed herein. In some embodiments, the method improves caspase activation. In some embodiments, the method sensitizes the cells to improve the response to a SMAC mimetic or BH3 mimetic or other pro-apoptotic drug or agent. The compound of Formula I or a salt thereof can be administered prior to, simultaneously with, or subsequent to the administration of the SMAC mimetic or BH3 mimetic or other pro-apoptotic drug. In some embodiments, the method includes contacting the compound or agent with a cell in vitro. In some embodiments, the method includes contacting the compound or agent with a cell in vivo or administering the compound or agent to a subject.

In some embodiments of any method disclosed herein, the compound of Formula I or a salt thereof in the combination is in an effective amount to decrease cell viability by about 10%, about 20%, about 30%, about 40%, about 50%, about 60%, about 80%, or about 100% in comparison with a vehicle alone. In some embodiments, the compound of Formula I or a salt thereof in the combination is in an effective amount to increase caspase 3/7 activation by about 10%, about 20%, about 30%, about 40%, about 50%, about 60%, about 80%, or about 100% in comparison with a vehicle alone. In some embodiments of any method disclosed herein, the compound of Formula I or a salt thereof in the combination is in an effective amount to sensitize cells to apoptosis/cell death to other pro-apopotic molecules (e.g. SMAC mimetic or BH3 mimetic, etc) by about 10%, about 20%, about 30%, about 40%, about 50%, about 60%, about 80%, or about 100% in comparison with a vehicle or the pro-apopotic molecule.

### Administration Regimen

The compound of Formula I, or a pharmaceutically acceptable salt thereof or a pharmaceutically composition thereof for the methods or kit described herein described herein may be administered to the subject by any suitable means. Non-limiting examples of methods of administration include, among others, (a) administration though oral pathways, which administration includes administration in capsule, tablet, granule, spray, syrup, or other such forms; (b) administration through non-oral pathways such as rectal, vaginal, intraurethral, intraocular, intranasal, or intraauricular, which administration includes administration as an aqueous suspension, an oily preparation or the like or as a drip, spray, suppository, salve, ointment or the like; (c) administration via injection, subcutaneously, intraperitoneally, intravenously, intramuscularly, intradermally, intraorbitally, intracapsularly, intraspinally, intrasternally, or the like, including infusion pump delivery; as well as (d) administration topically; as deemed appropriate by those of skill in the art for bringing the active compound into contact with living tissue.

Advantageously, the compound of Formula I, or a pharmaceutically acceptable salt thereof or a pharmaceutically composition thereof for administrations described above are prepared into dosage forms in a unit dose suited to fit a dose of the active ingredients. Such dosage forms in a unit dose include, for example, tablets, pills, capsules, injections (ampoules), suppositories, etc.

In exemplary embodiments of the pharmaceutical composition of the compound of Formula I, or a pharmaceutically acceptable salt thereof for oral administration, the composition can be a tablet, coated tablet, capsule, caplet, cachet, lozenges, gel capsule, hard gelatin capsule, soft gelatin capsule, troche, dragee, dispersion, powder, granule, pill, liquid, an aqueous or non-aqueous liquid suspension, an oil-in-liquid or oil-in-water emulsion, including sustained release formulations that are known in the art. For pediatric and geriatric applications, suspensions, syrups and chewable tablets are especially suitable.

The therapeutically effective amount (dosage) of the compound of Formula I, or a pharmaceutically acceptable salt thereof required will depend on the route of administration, the species (human or animal), and the physical characteristics of the particular subject or patient being treated. The dose can be tailored to achieve a desired effect, but will depend on such factors as weight, diet, concurrent medication and other factors which those skilled in the medical arts will recognize. More specifically, a therapeutically effective amount means an amount of compound effective to prevent, alleviate or ameliorate symptoms of disease or prolong the survival of the patient or animal being treated. Determination of a therapeutically effective amount is well within the capability of those skilled in the art, especially in light of the detailed disclosure provided herein.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be about 10 μg/kg to about 100 mg/kg body weight, preferably about 100 μg/kg to about 10 mg/kg body weight. Alternatively, dosages may be based and calculated upon the surface area of the animal, as understood by those of skill in the art.

The exact formulation, route of administration and dosage for the pharmaceutical compositions can be chosen by the individual physician in view of the patient's condition. (see e.g., Fingl et al. 1975, in “The Pharmacological Basis of Therapeutics”, which is hereby incorporated herein by reference in its entirety, with particular reference to Ch. 1, p. 1). In some embodiments, the dose range of the compound of Formula I or a pharmaceutically acceptable salt thereof administered to the subject or patient can be from about 0.5 to about 1000 mg/kg of their body weight. The dosage may be a single one or a series of two or more given in the course of one or more days, as is needed by the patient. In instances where human dosages for compounds have been established for at least some conditions, those same dosages, or dosages that are about 0.1% to about 500%, more preferably about 25% to about 250% of the established human dosage may be used.

It should be noted that the attending physician would know how to and when to terminate, interrupt, or adjust administration due to side-effects, toxicity or organ dysfunctions. Conversely, the attending physician would also know to adjust treatment to higher levels if the clinical response was not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency will also vary according to the age, body weight, and response of the individual patient. A program comparable to that discussed above may also be used in veterinary medicine.

Although the exact dosage will be determined on a drug-by-drug basis, in most cases, some generalizations regarding the dosage can be made. The daily dosage regimen for an adult human patient may be, for example, a peroral dose of about 0.01 mg to 2000 mg of the active ingredient, preferably from about 0.01 mg to about 500 mg. In other embodiments, an intravenous, subcutaneous, or intramuscular dose of the active ingredient of about 0.01 mg to about 100 mg, preferably about 0.01 mg to about 60 mg is used. In cases of administration of a pharmaceutically acceptable salt, dosages may be calculated as the freebase. In some embodiments, the composition is administered 1 to 4 times per day. Alternatively, a compound of Formula I or a pharmaceutically acceptable salt thereof may be administered by continuous intravenous infusion, preferably at a dose of up to about 1000 mg per day. As will be understood by those of skill in the art, in certain situations it may be necessary to administer a compound of Formula I or a pharmaceutically acceptable salt thereof disclosed herein in amounts that exceed, or even far exceed, the above-stated, preferred dosage range in order to effectively and aggressively treat particularly intractable diseases or conditions. In some embodiments, a compound of Formula I or a pharmaceutically acceptable salt thereof will be administered for a period of continuous therapy, for example for a week or more, or for months or years.

In some embodiments, a compound of Formula I or a pharmaceutically acceptable salt thereof is formulated into a dosage form for release for a period of 1 to 12, typically 3 to 12 hours, more typically 6-12 hours after administration. In some embodiments, the oral pharmaceutical compositions described herein may be administered in single or divided doses, from one to four times a day. The oral dosage forms may be conveniently presented in unit dosage forms and prepared by any methods well known to those skilled in the art of pharmacy.

A compound of Formula I or a pharmaceutically acceptable salt thereof can be evaluated for efficacy and toxicity using known methods. For example, the toxicology of the compound may be established by determining in vitro toxicity towards a cell line, such as a mammalian, and preferably human, cell line. The results of such studies are often predictive of toxicity in animals, such as mammals, or more specifically, humans. Alternatively, the toxicity may be determined in an animal model (such as mice, rats, rabbits, or monkeys) using known methods. The efficacy of a particular compound may be established using several recognized methods, such as in vitro methods, animal models, or human clinical trials. Recognized in vitro models exist for nearly every class of condition. Similarly, acceptable animal models may be used to establish the efficacy of chemicals to treat such conditions. When selecting a model to determine efficacy, the skilled artisan can be guided by the state of the art to choose an appropriate model, dose, and route of administration, and dosing regime. Of course, human clinical trials can also be used to determine the efficacy of a compound of Formula I or a pharmaceutically acceptable salt thereof in humans.

A compound of Formula I or a pharmaceutically acceptable salt thereof may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of Formula I or a pharmaceutically acceptable salt thereof formulated in a compatible pharmaceutical carrier may also be prepared, placed in an appropriate container, and labeled for treatment of an indicated condition.

Another aspect provides a method of screening for an inhibitor of mitofusin-mediated mitochondrial fusion. Through inhibition of mitofusin 1 and/or mitofusin 2, the compound inhibits mitofusin-mediated mitochondrial fusion and promotes mitochondrial fission. The method includes the following steps:
(i) providing a pharmacophore, wherein the pharmacophore comprises the structure:

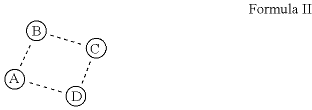

wherein:
A and B are each a hydrophobic moiety,
C is an aromatic moiety,
D is a hydrogen bond acceptor or a hydrogen bond acceptor
the distance (AB) between A and B is 4.0 angstrom,
the distance (BC) between B and C is 7 angstrom,
the distance (CD) between C and D is 3 angstrom,
the distance (AD) between A and D is 10 angstrom,
∠BAD is 62°,
∠ABD is 91°,
∠DBC is 17°, and
∠BDC is 51°,
(ii) obtaining a compound that resembles the pharmacophore as a candidate inhibitor of mitofusin-mediated mitochondrial fusion; and
(iii) optionally testing the compound in an assay for inhibitory activity against mitofusin-mediated mitochondrial fusion;
   wherein the compound that demonstrates inhibitory activity is an inhibitor of mitofusin 1 and/or mitofusin 2 for inhibition of mitofusin-mediated mitochondrial fusion.

In some embodiments,
C is an aromatic moiety,
D is a hydrogen bond acceptor or a hydrogen bond acceptor
the distance (AB) between A and B is 4.0 angstrom,
the distance (BC) between B and C is 6.7 angstrom,
the distance (CD) between C and D is 2.8 angstrom,
the distance (AD) between A and D is 9.8 angstrom,
∠BAD is 61.92°,
∠ABD is 91.38°,
∠DBC is 17.13°, and
∠BDC is 50.68°.

A library of compounds can be efficiently screened to identify one or more candidate inhibitors of mitofusin-mediated mitochondrial fusion. The qualify as a candidate, a compound also needs to have two hydrophobic moieties (A′ and B′), an aromatic moiety (C′) and a hydrogen bond acceptor or a hydrogen bond acceptor (D′) as those components of the pharmacophore of Formula I. The compound resembles the pharmacophore when the distances (A′B′, B′C′, C′D′, and A′D′) and angles (∠B′A′D′, ∠A′B′D′, ∠D′B′C′, and ∠B′D′C′) are within a pre-dertermined range of the respective distances (AB, BC, CD, and AD) and angles (∠BAD, ∠ABD, ∠DBC, and ∠BDC) in the pharmacophore. In some embodiments, the pre-determined range for each of the distances and angles is independently about 1%, about 3%, about 5%, about 8%, about 10%, about 12%, about 15%, about 20%, about 25%, about 30%, about 40%, or about 50%.

The distance is generally measured between atoms, between the centers of two groups or between an atom and the center of a group. The center of a ring is easily determined. In some cases of nonsymetrical groups, the center of a group is the center of a sphere that encloses the group. Preferably, the sphere can not be further reduced without exposing a portion of the enclosed group. However, depending on the specific groups, a flexibility tolerance of 0.2, 0.4, 0.6, 0.8, 1.0, 1.2, 1.5 or 2 Angstrom in the radius of the sphere is acceptable.

Hydrophobic group or moiety decreases a compound's solubility in water. Nonlimiting examples include alkyl (e.g C₁₋₁₀alkyl, branched or straight chained), haloC₁₋₁₀alkyl (e.g. mono, di, or tri halo-substituted C₁₋₁₀alkyl), 3-10 membered cycloakyl group, optionally substituted 6-10 membered aryl, and optionally substituted 5 to 14 membered heteroaryl. In some embodiments, the hydrophobic moiety for comparison purpose with the pharmacophore is an alkyl, an alkyl substituent on 3-10 membered ring (not the 3-10 membered ring), an optionally substituted 6-10 membered aryl or an optionally substituted 5 to 14 membered heteroaryl. Nonlimiting examples of the optional substituents include alkyl, halogen, 6-10 membered aryl and 5 to 14 membered heteroaryl.

Assays that can be used to monitor compound-induced inhibition of mitochondrial fusion in a mitofusin dependent manner include, for example, fluorescent microscopy to monitor mitochondrial shape and measurement of mitochondrial aspect ratio, EM microscopy to monitor mitochondrial shape, PEG mitochondrial fusion assay, and In vitro mitochondrial fusion luminescent and fluorescent-based assays.

The following non-limiting examples serve to further illustrate the embodiments of the present disclosure.

### EXAMPLES

Reagents. MASM7 was obtained from Enamine (cat. #EN300-396282). Screened MASMs were purchased from Enamine, ChemBridge and ChemDiv. MFI8 was obtained from ChemBridge (cat. #7681311) and also synthesized in house in a more stable form as a HCl salt. MFI8 was freshly dissolved in DMSO at 10 mM prior each experiment. MFI22-26 were also synthesized in house as HCl salts. The rest of the screened MFIs were purchased from Enamine, ChemBridge, ChemDiv, Vitas M and UORSY. All compounds were >95-98% pure, dissolved in 100% DMSO to prepare a 10 mM stock solution and diluted in aqueous buffers or cell culture medium for assays. BV6 (B4653) and Q-VD-Oph (A1901) were purchased from APExBio. Staurosporine (S1421) was purchased from Selleck. Myxothiazol (T5580) was purchased from Sigma. Rotenone was purchased from Tocris (36165).

Cell lines. Cell lines were purchased from ATCC. MEFs (WT, MENI KO, MFN2 KO and MFN1/MFN2 DKO) were also provided by David Chan's laboratory. DRP1 flox/flox MEFs were provided from Richard Kitsis' laboratory. U2OS cells were provided from Stephen Tait's laboratory. All cells maintained in DMEM (Life Technologies) supplemented with 10% FBS, 100 U ml 1 penicillin/streptomycin and 2 mM L-glutamine.

Mice. All animal experiments were approved by and performed in compliance with the guidelines and regulations approved by the Institutional Animal Care and Use Committee of the Albert Einstein College of Medicine.

Structural model of MFN2. The structural model of MFN2 was calculated based on the I-TASSER (Iterative Threading ASSEmbly Refinement) hierarchical approach to protein structure as we previously described and truncated crystal structure of MFN2 (PDB ID: 6JFL). Energy minimization and analysis of the top-ranked structure was performed with MAESTRO tools (Maestro, Schrödinger Release 2016-2020, Schrödinger, LLC). PyMOL (The PyMOL Molecular Graphics System. Schrödinger Release 2016-2020, Schrödinger, LLC) was used for preparing the figures.

Molecular dynamics simulations. Molecular dynamics (MD) simulations of the full-length MFN2 model structure was performed using DESMOND (Schrodinger LLC, 2021). Three independent 250 ns MD simulations were performed in a truncated TIP3P water box using OPLS3 force field and the NPT ensemble at 300K and constant pressure of 1.0325 bar. The Nosc-Hoover Chain thermostat and Martyna-Tobias-Klein barostat were used to maintain the temperature and pressure, respectively. The system was neutralized by Na+ and Cl− ions at a final concentration of 0.15 M. Before production runs, the system was minimized and pre-equilibrated using the standard equilibration protocol implemented in DESMOND. Analysis of the trajectories was performed using MAESTRO simulation event analysis tools (Schrodinger LLC, 2021). Interatomic distance plots obtained from MAESTRO were plotted using GraphPad Prism 9. PyMOL (Schrodinger LLC, 2021) was used to show structures of the MD trajectory snapshots.

In silico small molecule library preparation. eMolecules (www.emolecules.com) library of purchasable compounds was converted to 3D structures using LIGPREP (LigPrep, Schrödinger Release 2016, Schrödinger, LLC) and EPIK (Epik, Schrödinger Release 2016, Schrödinger, LLC) generating an in silico library of approximately 13.8 million compounds containing compounds with different ionization state at pH 7.0±2.0, stereochemistry and tautomeric form, excluding potential Pan Assay Interference Compounds (PAINS) using PAINS definitions included in Canvas. Conformation analysis of ligands was calculated using the OPLS3 force field.

3D Pharmacophore model generation and screen. Phase (Phase, Schrödinger Release 2016, Schrödinger, LLC) module was used to generate a pharmacophore hypothesis and a 3D pharmacophore screen. The coordinates of the HR1 helix residues Val372, Met376 and His380 from the structural model of MFN2 were used to assign pharmacophore points in 3D coordinates. Pharmacophore hypothesis included 5 features as defined in Phase for 3 hydrophobic groups to mimic the sidechain residues of Val273 and Met376 and an aromatic ring with a hydrogen-bond donor to mimic the sidechain of His380. The pharmacophore screen used the in silico library of compounds prepared from the eMolecules library in pre-existing conformations with the requirement to satisfy at least 4 out of the 5 pharmacophore features of the hypothesis. The top 1000 compounds ranked based on the Phase Score were selected for further visual analysis and clustered for diversity using dendritic fingerprints in Canvas. Physicochemical and AMDET properties including Lipinski rules, permeability, log P, metabolic liabilities and hERG inhibition were evaluated using QikProp (QikProp, Schrödinger Release 2016, Schrödinger, LLC). The highest 8 ranked compounds and the 10 most diverse compounds yielded selected molecules for experimental validation. MASM7 and MFI8 were checked for potential Pan Assay Interference Compounds (PAINS) and has not been reported as a hit in previous screens in Pubchem database.

Live cell imaging. MEFs were seeded on chamber slides (MatTek Corporation: 35 mm dishes, No. 1.5, 14 mm glass diameter) at ˜70% confluence. Cells were treated with MASM7 or MFI8. Then, cells were stained with MitoTracker Green (200 nM, Invitrogen) for 20 min at 37° C. After treatments, media was replaced with FluoroBrite™ DMEM (Invitrogen) supplemented with 10% FBS, 100 U ml 1 penicillin/streptomycin, 2 mM L-glutamine and MASM7 or MFI8 prior image acquisition. Images were taken with Leica SP5 inverted confocal microscope. Data were analyzed with Image J software. >200 mitochondria were measured per condition in mitochondrial aspect ratio.

Measurement of mitochondrial aspect ratio (Mito AR). Images that depict stained mitochondria with mitotracker green were analyzed using FIJI We measured for each mitochondrion its length and width. Then, we divided the length by the width for each mitochondrion to get its mitochondrial aspect ratio. >200 mitochondria were measured per treatment.

Immunofluorescent analysis and microscopy. Cells were treated with MASM7, MFI8 and or Q-VD-OPh at ˜70% confluence for 6 h. After treatments, cells were washed with PBS and fixed with 4% PFA for 15 min. Then cells were permeabilized with 0.1% Triton X-100 in PBS for 10 min. Cells were blocked with 5% BSA in PBS-T and incubated overnight with primary antibodies as indicated; yH2AX (Sigma; 05-636, 1:100 dilution). After incubation with primary antibodies, cells were washed with PBS and incubated with the appropriate mouse secondary antibody (ThermoFisher Scientific; A-11001) in the blocking solution. After PBS washes, coverslips were dipped in water and mounted on glass slides using Vectashield containing DAPI (Vector laboratories). Images were taken with Leica SP5 inverted confocal microscope. Data were analyzed with ImageJ.

Mutagenesis. Mutagenesis on the HR2 domain for the NMR analysis or the MFN2 gene that was used for the packaging of the adenovirus was performed by using QuikChange Lightning Site-Directed mutagenesis Kit (Qiagen; Cat. 210518). The following primers were used:

S685A (adeno):
(SEQ ID NO: 1)
5′-CTGGCTCCAACTGCGCCCACCAAGTCCAGC-3′
L692A (adeno):
(SEQ ID NO: 2)
5′-CCAAGTCCAGCAGGAAGCGTCTGGGACCTTTGC-3′
S685A (NMR):
(SEQ ID NO: 3)
5′-TGGGTAGCAACTGCGCCCACCAGGTGCAGC-3′
L692A (NMR):
(SEQ ID NO: 4)
5′-AGGTGCAGCAAGAGGCGAGCGGCACCTTCG-3′
D725A/L727A:
(SEQ ID NO: 5)
5′-AAGAAAATTGAAGTTCTGGCCAGCGCGCAAAG
CAAGGCGAAACTG-3′
L727A:
(SEQ ID NO: 6)
5′-AATTGAAGTTCTGGACAGCGCGCAAAGCAAGG
CGAAACT-3′

Recombinant HR2 protein production. Human MFN2 residues 678-757 corresponding to the HR2 domain were cloned into a pET-28 vector fused to a His-tag and transformed into BL21 (DE3) CodonPlus (DE3)-RIPL E. coli cells. Cells were grown at 37° C. in 1 L of LB media to an OD₆₀₀ of 0.8, cells were then harvested and resuspended in 1 L of Luria Broth media or M9 media supplemented with 1.5 gr/L of 15N ammonium chloride grown for 45 min at 37° C. and induced at 18° C. for 16 hours with 1 mM isopropyl B-d-1-thiogalactopyranoside. MFN2-HR2 or ¹⁵N-MFN2-HR2 domain was purified from bacterial pellets by high-pressure homogenization in lysis buffer (20 mM Tris·HCl pH 7, 250 mM KCl, 25 mM imidazole, and Roche complete EDTA free protease inhibitor cocktail) and ultracentrifuged at 45,000 g for 45 min. The supernatant was applied to pre-equilibrated 1 mL HisPur Ni-NTA Resin washed in lysis buffer and eluted using elution buffer (20 mM Tris·HCl pH 6, 250 mM KCl, 400 mM imidazole). MFN2-HR2 or 15N-MFN2-HR2 was further purified by size exclusion chromatography (Superdex 75 Increase 10/300 GL column) in gel filtration buffer (20 mM potassium phosphate pH 6, 150 mM KCl). Fractions containing the MFN2-HR2 domain were confirmed by SDS-PAGE, pooled and concentrated to 50 M in NMR buffer (20 mM potassium phosphate pH 6, 150 mM KCl, 10% D₂O) using a 10 KDa cut-off Centricon spin concentrator (Millipore) for prompt use in biochemical and NMR studies.

NMR experiments. The uniformly 15N-labeled protein samples were prepared by growing the bacteria in a minimal medium, as described above. Correlation ¹H-¹⁵N-HSQC spectra of 50 μM MFN2-HR2 in the presence and absence of MASM7 or MFI8 or 367-384Gly or 398-418Gly were recorded on a BRUKER AVANCE IIIHD 600 MHz system equipped with a 5 mm H/F-TCI CryoProbe at 25° C. All experiments were performed using an independent sample for each experimental measurement as a 400 μL sample in a 5-mm Shigemi; all samples were DMSO matched with 2% d₆-DMSO. Spectra were processed using qMDD (mddnmr v2.0) and NMRPIPE and analyzed using Analysis (CCPNMR).

Microscale Thermopheresis. Freshly purified His-tagged MFN2-HR2 domain was used for Microscale Thermophoresis (MST) binding studies. For protein labelling, a fresh stock of 5 μM His-tag-RED-tris-NTA 2^{nd} generation dye (Nanotemper) in 25 mM Hepes pH 7.5, 100 mM NaCl, 0.005% Tween-20 (assay buffer) was used to label 500 nM of MFN2-HR2 in the same buffer. The labeling reaction was incubated for 30 min at RT and centrifuged at 15.000×g at 4° C. for 10 min. Labelled protein from the supernatant was kept on ice and used immediately. Compounds were serially diluted in 100% DMSO. Immediately before mixing with labeled protein, the dilution series was transferred in assay buffer to reach 2% DMSO. 10 μl of each compound in the final series was mixed with 10 μl of labeled protein and samples were incubated for 5 min at 30° C. before MST measurement using a MonolithNT.115 instrument (Nanotemper). Peptides were diluted in assay buffer plus 2% DMSO and treated as above. Final MST conditions: 25 mM Hepes pH 7.5, 100 mM NaCl, 0.005% Tween-20, 1% DMSO, 125 nM labeled HR2, 25 nM NTA dye, temperature 30° C., LED power 40%, MST power Medium, before MST 3 sec, MST-on, 10 sec, after MST: 1 sec. Samples were measured in standard capillaries (Nanotemper) and showed no aggregation according to post-run analysis using the Monolith data collection software (Nanotemper). Kd values were obtained from non-linear regression fits of normalized data to a three or four parameter logistic curve, using GraphPad Prism 9.

In vitro MFN2 oligomerization. MEFs were seeded in 8×15 cm2 dishes and grown at ˜90% confluence. Then, mitochondria were isolated according to previously published protocol. Briefly, cells were harvested, pelleted and washed with cold PBS. Then, cells were resuspended in cold mitochondrial isolation buffer (0.2 M sucrose, 10 mM Tris-MOPS pH 7.4, 1 mM EGTA, 5 mM Mg(OAc)₂, 50 mM KOAc, 1×HALT protease inhibitors, 0.5 mM PMSF) and homogenized in dounce homogenizer with 20 strokes. Isolated mitochondria were incubated with 2 mM GTP, 10 μM MASM7 and 40 μM MFI8 at 37° C. for 30 min. Equal volume of 2× lysis buffer was added to each reaction to have final concentration of (50 mM Bis-Tris, 50 mM NaCl, 10% Glycerol, and 1% wt/vol Digitonin), then samples were incubated on ice for 15 min. Lysates were centrifuged at 16,000 g at 4° C. for 30 min. Subsequently, supernatant was mixed with NativePAGE 5% G-250 Sample Additive to a final concentration of 0.25%. Proteins were resolved with a Novex NativePAGE 4-16% Bis-Tris Protein Gels (Invitrogen) at 4° C. according to previously published protocol.

Blue native PAGE. The procedure for the Blue native PAGE (BN-PAGE) electrophoresis was adapted from previously published protocol. Lysates from the in vitro MFN2 oligomerization experiment were resolved with a Novex Native PAGE 4-16% Bis-Tris Protein Gels (Invitrogen) at 4° C. The gels were firstly run in the dark cathode buffer at 40 V for 30 min and then at 100 V for 30 min. Then, the dark cathode buffer was replaced with light cathode buffer and the gels were run at 100 V for 30 min and at 200 V for 1 hr and 50 min. Next, the gels were transferred to polyvinylidene fluororide (PVDF) membranes at 30 V for 16 h using a transfer buffer (Tris 25 mM, 192 mM glycine, 20% methanol). Once the transfer was completed, the membranes were incubated 8% acetic acid for 15 min and subsequently washed with water for 5 min. Then, membranes were dried at 37° C. for 20 min, rehydrated in 100% methanol, and washed with water. Finally, membranes were blocked for 1 hr in PBS-T containing 5% milk and 1% BSA and incubated overnight with an anti-MFN2 antibody (Cell Singaling; #9482S) at a 1:1000 dilution.

Cellular extract thermal shift assay (CETSA). MEFs were seeded in 8×15 cm² dish and grown at ˜90% confluence. The media was then removed and cells were washed with PBS. Cells were harvested using a cell scraper and centrifuged at 1200 rpm in 4° C. for 3 min. Then, cells were resuspended in 4 ml PBS. A suspension of 1 ml cells was treated with MASM7 (100 μM), MFI8 (100 μM), the combination of both compounds or DMSO (100 μM) and left rotating for 1 hr at room temperature. Upon completion of the treatment, 50 μl of the suspension of cells were transferred to PCR tubes and heated in a Biorad C₁₀₀₀ Touch Thermal Cycler for 3 minutes using a temperature gradient (25, 37, 39, 42, 46, 51, 56, 59° C.). All cells were lysed by four cycles of freeze thawing using liquid nitrogen. Samples were then centrifuged at 2×10⁴ g for 15 minutes. Equal volumes of supernatants were run on 10-well 4-12% NuPAGE SDS-PAGE gels (Invitrogen) and analyzed by western blot. ACTIN, which is temperature insensitive under these conditions, served as loading control. Results were quantitated by densitometric analysis as described below.

Densitometric analysis. Densitometric data from western blot scanned films were obtained using Image Studio software (LI-COR). Data were corrected to loading control (total ACTIN) and normalized to the treated bands that correspond to 25° C. (100%) and blot backgrounds (0%). Tm values were obtained from non-linear regression fits of normalized data to a four-parameter logistic curve (4PL), using GraphPad Prism 8.

Western blot. Western blots were performed from whole cell lysates (WCL) prepared in lysis buffer containing 50 mM Tris-HCl pH 7.5, 1% Triton X-100, 150 mM NaCl, 5 mM MgCl₂, 1 mM EGTA and 10% glycerol in the presence of protease inhibitor cocktail (Roche). WCL were separated on a 4-12% NuPAGE MES gel (Invitrogen), transferred into a PVDF membrane, block for 1 hr and immunoblot with the corresponding anti-bodies.

Mitochondrial depolarization assay (mtΔΨ). MEFs (10⁴ cells/well) were seeded in a 96-well black plate and treated with MASM7 or MFI8 for 6 hrs. Following treatments, cells were stained with 250 nM TMRE (Sigma; Cat. 87917) for 20 min at 37° C. Subsequently, cells were washed with thrice with PBS. Fluorescence intensity was detected by a M100 microplate reader (TECAN, Ex: 540 nm/Em: 579 nm).

Mitochondrial respiration in cellulo. Mitochondrial oxygen consumption rates (OCR) were assessed using a XF24 Analyzer (Seahorse Biosciences, Billerica MA, USA). In brief, 3×10⁴ MEFs were cultured in a XF24 cell culture microplate containing DMEM supplemented with 10% fetal bovine serum. Cells were treated with MASM7 (1 μM) or MFI8 (20 μM)₆ hrs prior to OCR analysis. Mitochondrial respiration was assessed by the sequential addition of oligomycin (1 μM), carbonyl cyanide-4-(trifluoromethoxy)phenylhydrazone (FCCP, 2 μM) and rotenone (1 μM)-antimycin (1 μM) as previously reported. OCR was normalized to cell number or total protein content per well for each condition tested.

Mitochondrial respiration in isolated mitochondria. Mitochondria were isolated based on previously published protocol. Isolated mitochondria were treated with MFI8 (20 μM) for 30 min prior to OCR analysis. OCR was measured using the Mitocell (MT200), a Clarktype electrode from Strathkelvin instruments. Isolated mitochondria (50 μg) from murine cardiomyocytes were loaded into the 50 μL magnetically stirred respiration chamber containing EBm buffer (1 M sucrose, 0.01 M Tris/HCl, 1 M MgCl₂, 0.1 M EGTA/Tris, 2 mM KH₂PO₄, pH 7.4). Glutamate and malate were added to the vessel at final concentrations of 0.25 M and 0.125 M, respectively. Respiration was measured after the addition of substrates (glutamate and malate) and termed “state 2 respiration”. ADP was then added at a final concentration of 800 μM to measure “state 3 respiration.” Addition of oligomycin at final concentration 800 ng/ml was used to quench the reaction and measure proton leakage. Finally, FCCP was added at final concentration 200 nm to measure maximum (uncoupled) respiration.

Caspase-3/7 activation assay. Cells (5×10³ cells/well) were seeded in a 96-well white plate and treated with MASM7, MFI8. Caspase 3/7 activation was measured after 6 hr by addition of the Caspase-Glo 3/7 reagent according to the manufacturer's protocol (Promega). Luminescence was detected by a F200 PRO microplate reader (TECAN). Caspase assays were performed in at least triplicate and the data normalized to vehicle-treated control wells. Dilutions of MASM7 or MFI8 were performed using a TECAN D300e Digital Dispenser from 10 mM stocks.

Cytochrome C release. MEFs were seeded in a 10 cm² dish and grown at ˜70% confluence. Following 6 hrs treatment with MFI8, cells were harvested and lysed in digitonin buffer [20 mM Hepes, pH 7.2, 10 mM KCl, 5 mM MgCl₂, Im MEDTA, 1 mM EGTA, 250 mM sucrose, 0.025% Digitonin (from 5% w/v stock) and complete protease inhibitors (Roche Applied Science)] on ice for 10 min. Then, supernatants were isolated by centrifugation at 15,000×g for 10 min. Mitochondrial pellets solubilized in 1% Triton X-100/PBS for 1 hr at 4° C. Solubilized Pellets were subjected to a 14,000×rpm spin for 10 min. Samples were prepared for western blot analysis and separated by 4-12% NuPage (Life Technologies).

Cell viability assay. Cells (5×10³ cells/well) were seeded in a 96-well white plate and treated with serial dilutions of MASM7 or MFI8. Cell viability was assayed after 72 hrs by addition of CellTiter-Glo reagent according to the manufacturer's protocol (Promega). Luminescence was measured using a F200 PRO microplate reader (TECAN). Viability assays were performed in at least triplicate and the data normalized to vehicle-treated control wells. Dilutions of MASM7 or MFI8 were performed using a TECAN D300e Digital Dispenser from 10 mM stocks.

Cell death assay. Cells (2×10⁵ cells/well) were seeded in a 6-well plate and treated with the indicated drugs for 6 hours. Cells were dissociated using Accutase (Thermo Fisher; Cat. #00-4555-56) in order to avoid accidental exposure of phosphatidylserine on the outer plasma membrane. Cell death was evaluated with Dead Cell Apoptosis Kit with Annexin V Alexa Fluor™ 488 & Propidium Iodide (Thermo Fisher; Cat. #V13241) according to manufacturer's protocol. Data was acquired by BD LSRII flow cytometer system using BD FACSDiva software. Data was analyzed by FlowJo (BD).

Metabolomics. MEFs were treated with MASM7 and MFI8. Cells were harvested using a cell scraper and centrifuged at 1200 rpm in 4° C. for 3 min. Each cell pellet sample was suspended into 250 to 700 μL of 80% aqueous methanol in an Eppendorf tube. The samples were vortex mixed for 15 s and sonicated in an ice-water bath for 5 min, followed by centrifugal clarification at 15,000 rpm and 5° C. in an Eppendorf 5424R centrifuge. The clear supernatants were collected. A standard stock solution of TCA cycle carboxylic acids, NAD and NADH was prepared in 80% methanol as S1. This standard solution S1 was serially diluted 1 to 4 (v/v) with the same solvent to make standard solutions S2 to S10. 20 μL of each standard solution and an aliquot of the clear supernatant from each cell was mixed with 20 μL of an internal standard solution containing 9 ¹³C— or deuterium labeled analogues of the TCA cycle carboxylic acids (except isocitric acid), 20 μL of 200 mM 3-NPH solution and 20 μL of 150 mM of EDC solution. The mixtures were allowed to react at 30° C. for 30 min. After reaction, 120 μL of water was added to each solution. 10 μL of the resultant solutions was injected into a C₁₈ UPLC column to quantitate the TCA cycle carboxylic acids by UPLC-MRM/MS with (−) ion detection, according to the procedure we described in a publication.

Minority MOMP. U2OS cells (100,000) were seeded onto glass coverslips and transfected with 250 ng each of CytoGFP and Mito-mCherry as previously described 39. 16 h after transfection, cells were treated with compounds for 3 h and then fixed in 4% paraformaldehyde. Cells were imaged on a Zeiss LSM880 with Airyscan using a 63×1.4NA objective. Subsequent to acquisition, images were processed using the Airyscan processing function in ZEN software. Minority MOMP was quantified by manually scoring the co-localisation of CytoGFP and mito-mCherry.

Analysis of mitochondrial biogenesis-related genes by q-PCR. Total RNA was extracted by using RNeasy Mini Kit (Qiagen) followed by cDNA synthesis using the SuperScript IV VILO Master Mix (Thermo Scientific), both performed according to the manufacturer's protocol. q-PCR was performed using the following primers:

Rpl139 Forward:
(SEQ ID NO: 7)
CAAAATCGCCCTATTCCTCA
Rpl139 Reverse:
(SEQ ID NO: 8)
AGACCCAGCTTCGTTCTCCT
Mfn1 Forward:
(SEQ ID NO: 9)
GACCGAAGGGTCAGATGAAA
Mfn1 Reverse:
(SEQ ID NO: 10)
TCCAGCTCTGTGGTGACATC
Mfn2 Forward:
(SEQ ID NO: 11)
CCTGGATGCTGATGTGTTTG
Mfn2 Reverse:
(SEQ ID NO: 12)
CCAGCTCATCCACCAGAAAG
Ppargc1a Forward:
(SEQ ID NO: 13)
TGATGTGAATGACTTGGATACAGACA
Ppargc1a Reverse:
(SEQ ID NO: 14)
GCTCATTGTTGTACTGGTTGGATATG
Ppargc1b Forward:
(SEQ ID NO: 15)
GGGAAAGGGACCAGACATAATC
Ppargc1b Reverse:
(SEQ ID NO: 16)
GCGGAAGCAGATGGTAAGATAA

For the q-PCR reaction, Power SYBR Green master mix (Thermo Scientific) was used according to the manufacturer's instructions. Briefly, each reaction consisted of 10 ng cDNA, 5 μL Power SYBR Green master mix, 200 nM primers (forward and reverse), and RNase-free water up to 10 μL. q-PCR was performed on the ViiA 7 Real-Time PCR System (Thermo Scientific) with the following cycle parameter: 95° C. for 10 min, 40 cycles of 95° C. for 15 s, and 60° C. for 1 min. q-PCR products were analyzed by melting curves for unspecific products or primer dimer formation. Rpl39 was used as housekeeping gene and 2^{-ΔΔCT} method was applied to determine the relative mRNAs expression.

Determination of mtDNA to genomic DNA ratio by q-PCR. DNA was extracted using DNeasy Blood and Tissue kit (Qiagen) according to the manufacturer's protocol. q-PCR was used to determine the ratio of mitochondrial DNA (mtDNA) and genomic DNA (gn DNA). The following primers were used for q-PCR reaction:

mt DNA (Nd2) Forward:
(SEQ ID NO: 17)
CCTATCACCCTTGCCATCAT
mt DNA (Nd2) Reverse:
(SEQ ID NO: 18)
GAGGCTGTTGCTTGTGTGAC
gn DNA (Pecam1) Forward:
(SEQ ID NO: 19
ATGGAAAGCCTGCCATCATG
gn DNA (Pecam1) Reverse:
(SEQ ID NO: 20)
TCCTTGTTGTTCAGCATCAC

Each reaction consisted of 5 ng of DNA, 5 μL Power SYBR Green master mix (Thermo Scientific), 200 nM primers (forward and reverse), and RNase-free water up to 10 μL. q-PCR was performed on the ViiA 7 Real-Time PCR System (Thermo Scientific) at 95° C. for 10 minutes, 40 cycles of 95° C. for 15 s, and 60° C. for 1 minute, followed by melting curve analysis. 2^{-ΔΔCT} method was applied to determine the mtDNA/nDNA ratio, being ΔCT=CT (mtDNA gene)-CT (nDNA gene).

RNA-seq. Sequencing results were demultiplexed and converted to FASTQ format using Illumina bcl2fastq software. The sequencing reads were adapter and quality trimmed with Trimmomatic and then aligned to the mouse genome (build mm10/GRCm38) using the splice-aware STAR aligner. The featureCounts program was utilized to generate counts for each gene based on how many aligned reads overlap its exons. These counts were then normalized and used to test for differential expression using negative binomial generalized linear models implemented by the DESeq2 R package.

Chemical syntheses. All chemical reagents and solvents were obtained from commercial sources and used without further purification. FastWoRX™ was purchased from Faster Chemistry LLC. Microwave reactions were performed using an Anton Paar Monowave 300 reactor. Chromatography was performed on a Teledyne ISCO CombiFlash R_{f} 200i using disposable silica cartridges. Analytical thin layer chromatography (TLC) was performed on Merck silica gel plates and compounds were visualized using UV. NMR spectra were recorded on a Bruker 600 spectrometers. ¹H chemical and ¹³C chemical shifts (δ) are reported relative to tetramethyl silane (TMS, 0.00 ppm) as internal standard or relative to residual solvent signals. Mass spectra were recorded by the Proteomics Facility at the Albert Einstein College of Medicine.

### Synthesis of Compound 8 (MASM7)

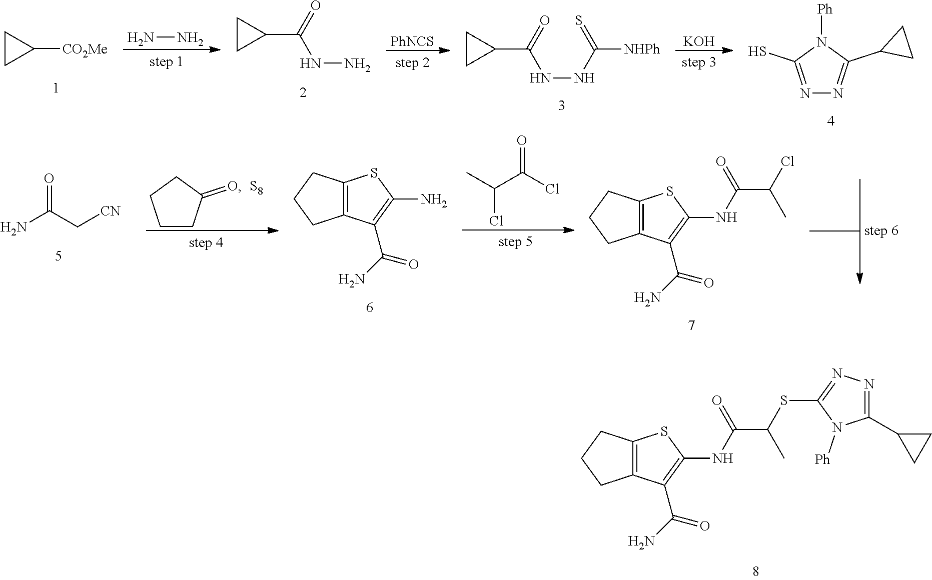

### Synthesis of Compound 4

Step 1. Cyclopropanecarbohydrazide (2): A mixture of methyl cyclopropanecarboxylate (26.31 g, 26.28 mmol) of 1 and hydrazine hydrate (30 g) was refluxed for 12 hours and then placed in vacuum desiccators over sulfuric acid for several days. The crude product was recrystallized from benzene containing a small amount of ethanol and yielded hydrazide 2 (22.4 g, 85%).

Step 2. 2-(cyclopropanecarbonyl)-N-phenylhydrazine-1-carbothioamide (3). Hydrazide 2 (2.08 g, 20.8 mmol) and phenyl isothiocyanate (2.16 g, 16.0 mmol) were dissolved in THF (80 mL), and the mixture was heated to reflux for 7 hours. After cooling, the insoluble product was collected by filtration and washed with water, yielding 3 (3.52 g, 94%).

Step 3. 5-cyclopropyl-4-phenyl-4H-1,2,4-triazole-3-thiol (4). A solution of potassium hydroxide (2.16 g (38.5 mmol) in water (50 mL) was stirred while 3 (3.50 g, 14.9 mmol) was added. The solution was warmed on a steam bath for 1 hour. After cooling, the solution was poured into a dilute hydrochloric acid solution. The insoluble product was collected by filtration and washed with water, giving 4 (2.36, g 73%).

¹H NMR (400 MHZ, DMSO-d6) δ 13.60 (s, 1H), 7.55 (m, 3H), 7.46 (d, 2H), 1.47 (m, 1H), 0.90 (m, 2H), 0.83 (m, 2H). 13C NMR (101 MHz, DMSO-d6) δ 168.06, 154.14, 134.28, 129.83, 128.80, 7.47, 6.73. APSI MS: calculated for C₁₁H₁₂N₃S (M+H)+218.1 found 218.2.

### Synthesis of Compound 8

Step 4. 2-amino-5,6-dihydro-4H-cyclopenta[b]thiophene-3-carboxamide (6). To the stirred solution of cyclopentanone (25.71 g, 306 mmol), 2-cyanoacetamide 5 (25.0 g, 297 mmol), sulfur powder (9.80 g, 306 mmol) in ethanol (400 ml) was added morpholine (53.3 mL, 618 mmol) and the reaction mixture was stirred at room temperature for 6 hours. The reaction mixture was concentrated, diluted with EtOAc and washed with H₂O (2×300 mL). The separated organic layer was dried over anhydrous Na₂SO₄, evaporated and crystallized from ethanol to get compound 6 (29.52 g, 53%).

Step 5. 2-(2-chloropropanamido)-5,6-dihydro-4H-cyclopenta[b]thiophene-3-carboxamide (7). To a mixture of 6 (3.00 g, 16.5 mmol) and triethylamine (1.83 g, 18.1 mmol) in CH₂Cl₂ (20 mL) was added 2-chloropropionyl chloride (2.30 g, 18.1 mmol) slowly at 0° C., and the resulting mixture was stirred at room temperature for 2 hours. The reaction mixture was concentrated to half of the volume to afford the solid product, which was filtered, washed with CH₂Cl₂ (2 mL) and dried to provide compound 7 (3.68 g, yield 82%).

1H NMR (DMSO-d6, 400 MHZ) δ 12.67 (s, 1H), 7.66 (s, 1H), 6.74 (s, 1H), 4.97 (q, 1H), 2.92 (t, 2H), 2.80 (t, 2H), 2.35 (p, 2H), 1.67 (d, 3H). ¹³C NMR (101 MHZ, DMSO-d6) δ 167.45, 166.50, 147.80, 139.74, 132.83, 112.51, 55.12, 29.48, 28.73, 28.16, 22.04. APSI MS: calculated for C₁₁H₁₄ClN₂O₂S (M+H)⁺ 273.1 found 273.0.

Step 6. 2-(2-((5-cyclopropyl-4-phenyl-4H-1,2,4-triazol-3-yl)thio) propanamido)-5,6-dihydro-4H-cyclopenta[b]thiophene-3-carboxamide (8). To a mixture of compound 4 (3.30 g, 15.2 mmol) and KOH (0.940 g, 16.8 mmol) in methanol (100 mL), compound 7 (5.40 g, 19.8 mmol) was added and the reaction mass was vigorously stirred for 1 hour at 35° C. The volatiles were evaporated, and the residue was diluted with water. The product was extracted with dichloromethane. The organic extract was dried over Na₂SO₄ and evaporated under reduced pressure. The residue was purified by silica gel column chromatography to give the target compound 10 (MASM7, 2.50 g (5.51 mmol, 36%).

¹H NMR (DMSO-d6, 400 MHz)_{δ 0.91} (m, 2H), 1.48 (d, 3H), 1.54 (m, 1H), 2.34 (m, 2H), 2.78 (t, 2H), 2.89 (t, 2H), 4.35 (q, 1H), 6.66 (s, 1H), 7.53 (m, 6H), 12.35 (s, 1H). 13C NMR (101 MHz, DMSO-d6) δ 167.69, 167.34, 157.86, 148.26, 147.53, 139.61, 133.42, 132.38, 130.26, 127.88, 112.08, 45.32, 29.53, 28.74, 28.18, 18.14, 7.89, 6.25. APSI MS: calculated for C₂₂H₂₄N₅O₂S₂ (M+H)⁺ 454.1 found 454.2.

### Synthesis of 4-Chloro-2-(1-((2,3-dimethylphenyl)amino)ethyl)phenol

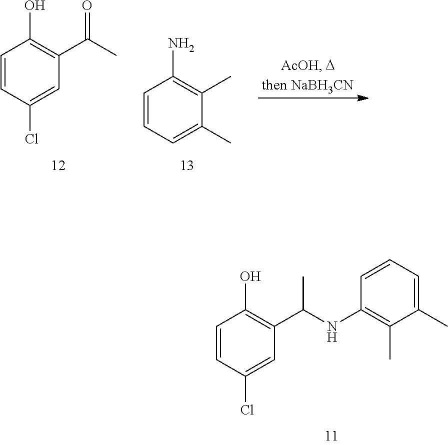

4-Chloro-2-(1-((2,3-dimethylphenyl)amino)ethyl)phenol (11 or MFI8): 1-(5-chloro-2-hydroxyphenyl) ethan-1-one (880 mg, 5.16 mmol), 2,3-dimethylaniline (940 mg, 7.74 mmol), acetic acid (4.8 mL) and methanol (1.2 mL) were combined in a microwave vial. The vial was capped and heated to 100° C. for 120 min. After cooling to room temperature. NaBH₃CN (486 mg, 7.74 mmol) was added and the mixture was stirred for 30 min. The reaction mixture was diluted with water (100 mL) and FastWoRX (4.5 g) was added along with CH₂Cl₂ (15 mL). The CH₂Cl₂ was then removed on rotavap then the solids were filtered and washed with water, aq. NaHCO₃, water, and finally air-dried. The dry polymer was loaded in a cartridge and the product was purified by column chromatography (12 g silica; 0-30% CH₂Cl₂ in hexanes). In some cases the product was contaminated with excess primary aniline at this stage. The impurity was removed by dissolving in minimum CH₂Cl₂ and adding hexanes to precipitate the product. The liquid phase was then removed and the solids washed with hexanes to give pure product. The fractions containing the product were collected and reduced to about half the volume before HCl (1 M in Et₂O; 10 mL) was added, causing precipitation of the HCl salt which was collected by filtration (440 mg, 1.41 mmol, 27%).

TLC: R_{f}=0.52 (Hexanes:CH₂Cl₂ 1:2; UV). 1H NMR (600 MHZ, D₂O): δ 7.24 (d, J=2.6 Hz, 1H), 7.23-7.17 (m, 2H), 7.06 (t, J=7.8 Hz, 1H), 6.95 (d, J=8.0 Hz, 1H), 6.78 (d, J=8.7 Hz, 1H), 4.90 (q, J=6.9 Hz, 1H), 2.21 (s, 3H), 2.20 (s, 3H), 1.65 (d, J=7.0 Hz, 3H). 13C NMR (151 MHz, D₂O): δ 153.34, 140.28, 131.42, 131.01, 130.73, 130.66, 128.66, 126.41, 124.59, 123.14, 121.81, 117.27, 58.19, 19.36, 15.71, 13.15. ESI-MS: calculated for C₁₆H₁₉ClNO (M+H)⁺ 276.1150 found 276.1150.

### Synthesis of 2-(1-((2,3-Dimethylphenyl)amino)ethyl)phenol (12 or MFI22)

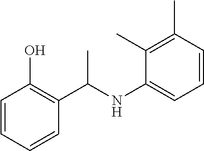 [SMILES: Cc1cccc(NC(C)c2ccccc2O)c1C]

2-(1-((2,3-Dimethylphenyl)amino)ethyl)phenol (MFI22): synthesized following the general procedure from 1-(2-hydroxyphenyl) ethan-1-one (200 mg, 1.47 mmol) and 2,3-dimethylaniline (240 mg, 1.98 mmol) yielding 32 mg (9%).

TLC: R_{f}=0.47 (Hexanes:CH₂Cl₂ 1:2; UV). 1H NMR (600 MHZ, D₂O) δ 7.34-7.25 (m, 3H), 7.13 (t, J=7.8 Hz, 1H), 7.04 (d, J=8.0 Hz, 1H), 6.94 (t, J=7.6 Hz, 1H), 6.92 (d, J=8.6 Hz, 1H), 4.99 (q, J=6.9 Hz, 1H), 2.29 (d, J=14.1 Hz, 6H), 1.73 (d, J=7.0 Hz, 3H). 13C NMR (151 MHz, D₂O) δ 154.43, 140.19, 131.59, 131.07, 130.92, 130.71, 128.99, 126.38, 121.78, 121.58, 120.62, 115.87, 58.77, 19.37, 15.89, 13.14. ESI-MS: calculated for C₁₆H₂₀NO (M+H)⁺ 242.1139 found 242.1538.

### Synthesis of 4-Chloro-2-(((2,3-dimethylphenyl)amino)methyl)phenol (MFI23)

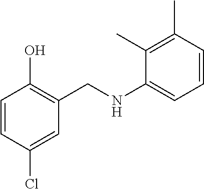 [SMILES: Cc1cccc(NCc2cc(Cl)ccc2O)c1C]

4-Chloro-2-(((2,3-dimethylphenyl)amino)methyl)phenol (13 or MFI23): synthesized following the general procedure from 5-chloro-2-hydroxybenzaldehyde (204 mg, 1.30 mmol) and 2,3-dimethylaniline (240 mg, 2.0 mmol) yielding 34 mg (10%).

TLC: R_{f}=0.46 (Hexanes:CH₂Cl₂ 1:2; UV). 1H NMR (600 MHZ, D₂O) δ 7.35-7.31 (m, 2H), 7.22 (t, J=7.8 Hz, 1H), 7.18 (d, J=2.7 Hz, 1H), 7.11 (d, J=8.0 Hz, 1H), 6.93 (d, J=8.7, 1H), 4.53 (s, 2H), 2.31 (s, 3H), 2.28 (s, 3H). 13C NMR (151 MHZ, D₂O) δ 154.25, 140.28, 132.39, 131.45, 131.23, 131.03, 130.39, 126.79, 124.14, 120.87, 118.48, 116.76, 50.74, 19.33, 12.73. ESI-MS: calculated for C₁₅H₁₇ClNO (M+H)⁺ 262.0993 found 262.0993.

### Synthesis of 4-Chloro-2-((m-tolylamino)methyl)phenol (MFI24)

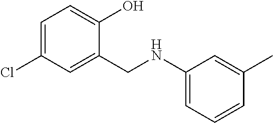 [SMILES: Cc1cccc(NCc2cc(Cl)ccc2O)c1]

4-Chloro-2-((m-tolylamino)methyl)phenol (14 or MFI24): synthesized following the general procedure from 5-chloro-2-hydroxybenzaldehyde (204 mg, 1.30 mmol) and m-toluidine (209 mg, 1.95 mmol) yielding 50 mg (16%).

TLC: R_{f}=0.38 (Hexanes:CH₂Cl₂ 1:2; UV). 1H NMR (600 MHZ, D₂O) δ 7.39 (t, J=7.8 Hz, 1H), 7.34 (d, J=7.7 Hz, 1H), 7.32 (dd, J=8.7, 2.6 Hz, 1H), 7.18 (s, 1H), 7.16-7.13 (m, 2H), 6.93 (d, J=8.7 Hz, 1H), 4.57 (s, 2H), 2.35 (s, 3H). ¹³C NMR (151 MHz, D₂O) δ 154.06, 140.97, 133.74, 131.40, 131.10, 130.49, 129.85, 124.12, 123.15, 119.60, 118.47, 116.75, 51.21, 20.22. ESI-MS: calculated for C₁₄H₁₅ClNO (M+H)⁺ 248.0837 found 248.0837.

### Synthesis of 4-Chloro-2-(1-(phenylamino)ethyl)phenol (MFI25)

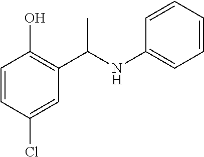 [SMILES: CC(Nc1ccccc1)c1cc(Cl)ccc1O]

4-Chloro-2-(1-(phenylamino)ethyl)phenol (15 or MFI25): synthesized following the general procedure from 1-(5-chloro-2-hydroxyphenyl) ethan-1-one (222 mg, 1.30 mmol) and aniline (182 mg, 2.0 mmol) yielding 22 mg (7%).

TLC: R_{f}=0.39 (Hexanes:CH₂Cl₂ 1:2; UV). ¹H NMR (600 MHZ, D₂O) δ 7.40-7.35 (m, 3H), 7.28-7.23 (m, 2H), 7.18 (dd, J=8.6, 2.5 Hz, 1H), 7.15 (d, J=2.6 Hz, 1H), 6.80 (d, J=8.7 Hz, 1H), 4.90 (q, J=6.9 Hz, 1H), 1.68 (d, J=7.0 Hz, 3H). 13C NMR (151 MHZ, D₂O) δ 153.12, 133.06, 130.52, 129.92, 129.85, 128.81, 124.56, 123.15, 123.03, 117.26, 59.30, 15.97. ESI-MS: calculated for C₁₄H₁₅ClNO (M+H)⁺ 248.0837 found 248.0836.

### Synthesis of N-(1-(3-chlorophenyl)ethyl)-2,3-dimethylaniline (MFI26)

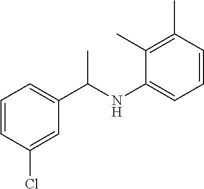 [SMILES: Cc1cccc(NC(C)c2cccc(Cl)c2)c1C]

N-(1-(3-chlorophenyl)ethyl)-2,3-dimethylaniline (16 or MFI26): synthesized following the general procedure from 1-(3-chlorophenyl) ethan-1-one (200 mg, 1.30 mmol) and 2,3-dimethylaniline (235 mg, 1.9 mmol) yielding 30 mg (9%).

TLC: R_{f}=0.31 (Hexanes:CH₂Cl₂ 1:2; UV). 1H NMR (600 MHZ, D₂O) δ 7.48 (m, 1H), 7.37 (t, J=7.9 Hz, 1H), 7.34 (t, J=1.9 Hz, 1H), 7.33 (d, J=7.5 Hz, 1H), 7.25 (d, J=7.8 Hz, 1H), 7.15 (t, J=7.8 Hz, 1H), 6.89 (d, J=8.0 Hz, 1H), 2.29 (s, 3H), 2.14 (s, 3H), 1.85 (d, J=6.9 Hz, 3H). NOTE: The signal from the benzylic proton is obscured by the solvent signal. 13C NMR (151 MHZ, D₂O) δ 140.42, 136.50, 134.15, 131.39, 131.08, 130.58, 130.47, 129.91, 128.30, 126.67, 126.52, 121.94, 62.35, 19.34, 16.90, 13.14. ESI-MS: calculated for C₁₆H₁₉ClN (M+H)⁺ 260.1201 found 260.1199.

### Synthesis of 5-chloro-N-(2,3-dimethylphenyl)-2-hydroxybenzamide (MFI27)

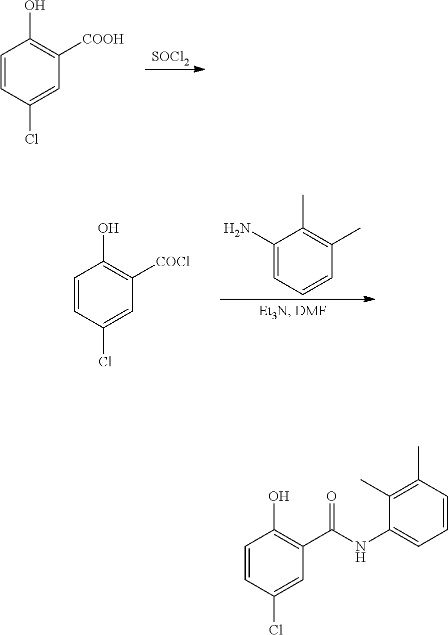

To a suspension of 5-chloro-2-hydroxybenzoic acid (500 mg, 2.90 mmol) in toluene (4 mL) was added thionyl chloride (4.0 mL). The resulting mixture was heated in 80° C. bath with stirring and turned to a clear solution. The heating was continued for 4 hours. The reaction mixture was cooled to r.t., concentrated by rotavap and high vacuum pump to remove most volatiles to afford crude acid chloride as white solid. Anhydrous dichloromethane (ca.5 mL) was added to the acid chloride, but it was not very soluble. Ethyl acetate (3˜5 mL) was added to the suspension until the acid chloride was completely dissolved.

To a solution of 2,3-dimethylaniline (354 μL, 2.90 mmol) in anhydrous dichloromethane (4 mL), was added triethyl amine (808 μL, 5.80 mmol). The resulting solution was cooled with ice bath. The acid chloride solution in DCM/EA was added via a syringe with stirring. The reaction mixture was allowed to warm up to r.t. and stirred overnight. The reaction mixture turned yellow with precipitation formation. The reaction mixture was concentrated by rotavap and diluted with ethyl acetate (8 mL). The resulting suspension was filtered, and the solid was rinsed with ethyl acetate (4 mL×2). The combined organics were concentrated and purified by chromatography on silica gel (hexane/ethyl acetate: 95:5 to 80:20). The desired compound was obtained as white solid, which was further purified by recrystalization (ethyl acetate/hexane) to afford analytical pure product (49 mg, 6%). Proton-NMR (600 MHZ, DMSO-d6): 12.29 (s, 1H), 10.34 (s, 1H), 8.05 (d, J=2.6 Hz, 1H), 7.47-7.50 (m, 2H), 7.12 (t, J=7.7 Hz, 1H)), 7.08 (d, J=7.4 Hz, 1H), 7.03 (d, J=8.8 Hz, 1H). C₁₃-NMR (600 MHZ, DMSO-d6): 165.32, 157.68, 137.51, 136.00, 133.71, 130.86, 129.04, 127.75, 125.96, 123.30, 123.27, 119.68, 119.07, 20.67, 14.33. ESI-MS (negative mode): 274 (M−1).

Statistical analysis. The results are presented as mean±SEM with a minimum of three replicates unless otherwise specified. Statistical analyses were performed by Student's 1-tests or one-way or two-way ANOVA using Prism 8.1 (GraphPad software). When the overall ANOVA revealed a significant effect, the data were further analyzed with the Dunnett, Sidak or Tukey post hoc test to determine specific group differences. P values indicated on the graphs: *p<0.05, **p<0.01, ***p<0.001, ****p<0.0001.

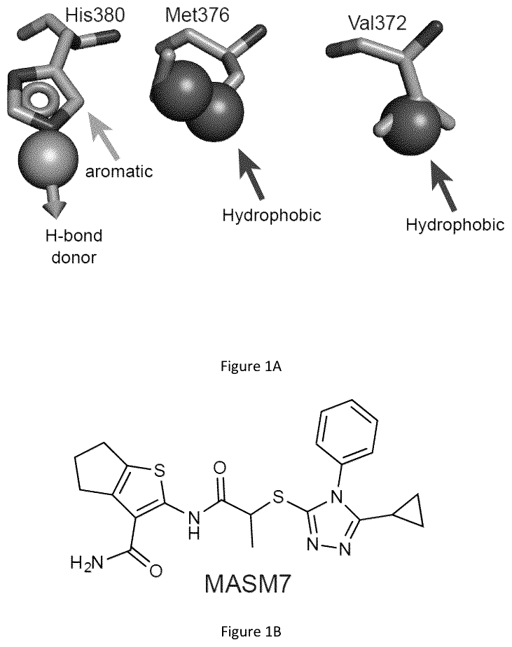

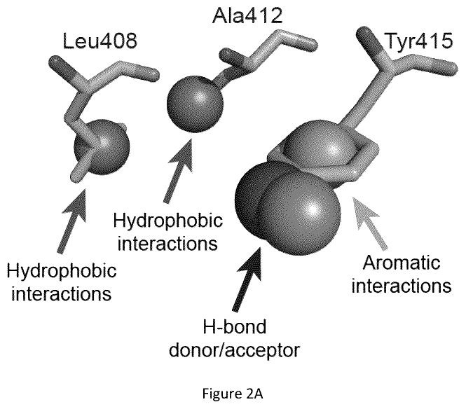

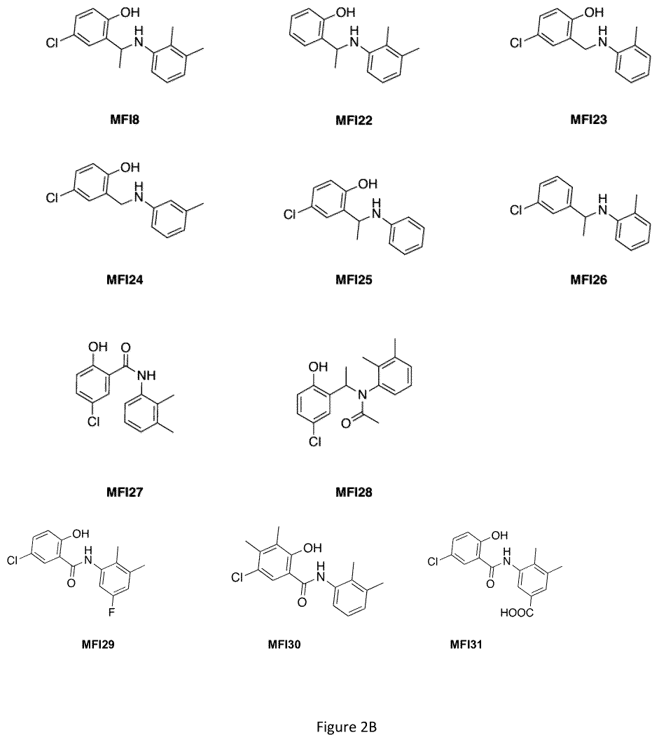

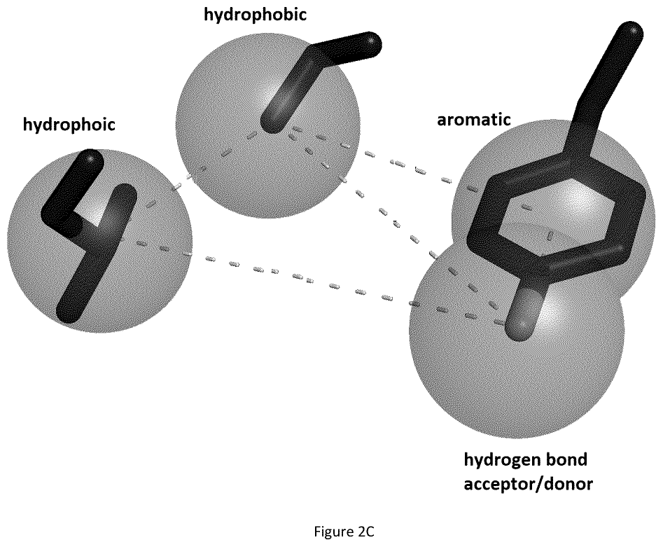

A small molecule inhibitor of mitochondrial fusion via mitofusins. Inter-molecular interactions of the HR2 domains of MFNs from adjacent mitochondria mediate MFNs oligomerization. It was therefore posited that a small molecule capable of inhibiting such inter-molecular HR2-HR2 interactions could act as an inhibitor of MFNs oligomerization and subsequently mitochondrial fusion. Structural analysis can be employed for the development a pharmacophore model, which is used in an in silico small molecule screening to identify small molecules that activate mitofusins. FIG. 1a shows a pharmacophore hypothesis based on the sidechains of the HR1-amino acids: Val372, Met376, His380 interacting with HR2 as in (C) comprising of 3 hydrophobic points, one aromatic ring and one hydrogen bond donor. FIG. 1b shows the chemical structure of MASM7. Previously a helical peptide from the MFN2 HR1 residues: 398-418 was found to inhibit mitochondrial fusion in cells by inhibiting HR2-HR2 inter-molecular interactions and subsequently MFNs oligomerization. This prompted visual inspection of the interactions of the HR1-residues 398-418 with the HR2 residues to gain structural insights for small molecule mimicry. Specifically, hydrophobic interactions were observed between the HR1-residues: Leu408, Ala412 and the HR2-residues: Leu692, Val688, and a possible hydrogen bond between the HR1-amino acid: Tyr415 and HR2-amino acid: Ser685. Of note, these HR1 residues are conserved among different species of MFN2 and MFN1. Hence, it was envisioned that a small molecule capable of recapitulating the interactions of the aforementioned HR1-amino acids would compete with inter-molecular HR2-HR2 interactions and inhibit MFNs oligomerization. Accordingly, to identify such a small molecule a strategy was adopted including virtual library preparation, pharmacophore screen with Phase, selection of top-ranked hits, interaction analysis and molecular property-based selection, and testing of selected hits experimentally. An in silico pharmacophore model was generated that screens small molecules to mimic specifically the side chains of the HR1 residues: Leu408, Ala412 and Tyr415 and bind to the corresponding HR2 residues. The pharmacophore model includes two hydrophobic interactions, an aromatic ring and a hydrogen bond donor/acceptor (FIG. 2a). Next the in silico library of 13.8×10⁶ commercially available small molecules was screened using the strategy described above. Likewise a set of 21 putative Mitochondrial Fusion Inhibitors (MFIs) was selected for experimental validation based on their fit to the pharmacophore model and molecular diversity of their scaffolds.

Selected hits were screened for their capacity to inhibit mitochondrial fusion in cells by monitoring mitochondrial morphology and using mitochondrial aspect ratio as a readout. Strikingly, MFI8 reduced significantly mitochondrial aspect ratio and emerged as the most effective compound in inhibiting mitochondrial fusion and subsequently promoting mitochondrial fission (FIG. 2b). Titration of MFI8 showed a concentration-dependent reduction of the mitochondrial aspect ratio. MFI8 has a small structure but possesses functional groups that could fulfill the 4 criteria of the pharmacophore model used for the in silico screen. Thus, the phenolic ring could participate in pi-stacking interactions and act as a hydrogen bond donor/acceptor, as the side chain of Tyr415, while the dimethyl-substituted phenyl ring could mimic the hydrophobic interactions of Leu408 and Ala412. To further validate that MFI8 meets the 4 criteria of the pharmacophore model hypothesis, structure activity relationships around the MFI8 scaffold were investigated. A series of MFI8 analogues were generated and evaluated for their capacity to promote mitochondrial fragmentation in cells. Substitution of the chlorine with hydrogen in the aromatic ring of MFI22 significantly reduced the capacity of the compound to promote mitochondrial fragmentation, suggesting that loss of chlorine affects the electron density of the aromatic ring and impairs its interaction. Loss of the methyl group in the aliphatic chain that connects the two phenyl rings had a minimal effect on the activity of MFI23. Furthermore, loss of each methyl group in the phenyl ring of MFI8 was not tolerated as MFI24 and MFI25 did not reduce mitochondrial aspect ratio, indicating the importance of those two methyl groups in mediating hydrophobic contacts for MFI8. Importantly, loss of the hydroxyl group of the phenolic ring was detrimental for the activity of MFI26, indicating that MFI8 forms a crucial hydrogen bond with the HR2 domain. Taken together, these data indicated that MFI8 possesses functional groups that meet the criteria of the pharmacophore model and specifically interact with mitofusins. A pharmacophore model of an inhibitor of mitofusin-mediated mitochondrial fusion is illustrated in FIG. 2c.

Next it was investigated whether MFI8 inhibited mitochondrial fusion in a MFNs dependent manner using mitochondrial aspect ratio as a readout. MFI8 was still capable of reducing mitochondrial aspect ratio when either MFN1 or MFN2 was knocked out. In contrast, double knockout of MFN1 and MFN2 completely abolished MFI8 from reducing mitochondrial aspect ratio. Collectively, these data indicate that MFI8 can promote mitochondrial fission by inhibiting mitochondrial fusion and interfering with the formation of either homotypic or heterotypic MFNs complexes. Importantly, MFI8 reduced mitochondrial aspect ratio even when it was co-treated with MASM7 in MEFs. Such a result supports the idea that MFI8 operates on the tethering permissive structure of MFNs and inhibits MFNs oligomerization by reducing the HR2-HR2 inter-molecular interactions.

Mitochondrial fusion positively correlates with mitochondrial respiration and membrane potential. Furthermore, inhibition of mitochondrial fusion decreases mitochondrial membrane potential and subsequently respiration. MASM7 increases mitochondrial functionality such as mitochodnrial respiration and membrane potential. MFI8 decreases mitochondrial functionality such as mitochondrial respiration and membrane potential. A series of mutagenesis experiments in vitro and in cellulo supported the observation that both MASM7 and MFI8 interact specifically with the HR2 domain of MFN2, albeit at different binding sites. Additional experiments from isolated mitochondria and in cells were used to demonstrate that both MASM7 and MFI8 modulate the conformational plasticity of MFN2 and its capacity to form oligomers. MASM7 promotes oligomerization of MFN2 by promoting the pro-tethering conformation of MFN2, while MFI8 directly inhibits the mitofusin oligomerization.

Inhibition of mitochondrial fusion promotes minority mitochondrial outer membrane permeabilization, and subsequently caspase 3/7 activation, albeit at sub-lethal levels. Aberrant mitochondrial fragmentation promotes minority MOMP and sub-lethal caspase 3/7 activation. Experiments have connected mitochondrial shape with mitochondrial membrane integrity and caspase 3/7 activation. Further experiments showed that MFI8 sensitizes cells to BV6 (SMAC mimetic) treatment in a mitofusin and APAF-1 dependent manner. Inhibition of mitochondrial fusion promotes minority MOMP and activates caspases 3/7, which can be used in combination with chemotherapeutic agents that act downstream of MOMP, such as Smac mimetics (e.g.BV6), to promote synergistic cell death in cells.

MFI8 binds to the HR2 domain of MFN2. To confirm that MASM7 and MFI8 directly interact with the HR2 domain, for the first time an 15N-labeled MFN2-HR2 domain (residues 678-757) was prepared and heteronuclear single quantum coherence (HSQC) spectra with or without MASM7 or MFI8 was recorded. HSQC NMR spectra of the recombinant HR2 domain showed evidence of a folded conformation of HR2. Titration of MASM7 induced peak broadening and shifting of select cross peaks of HR2 residues in the HSQC spectra, demonstrating direct binding to the HR2 domain. Similar cross peaks broadened and shifted upon titration of the 367-384Gly peptide, indicating that both MASM7 and 367-384Gly interact with similar HR2 residues. To further confirm that the interaction of MASM7 is specific to the expected binding region of HR2, a double mutant of HR2, D725A/L727A, was generated that should disrupt binding of MASM7 based on its proposed interaction with the HR2 domain. Titration of MASM7 to an 15N-labeled MFN2-HR2 D725A/L727A mutant under the same conditions showed lack of peak shifts or peak broadening indicating lack of MASM7 binding to the MFN2-HR2 D725A/L727A mutant.

Titration of MFI8 to the 15N-labeled MFN2-HR2 revealed peak broadening and shifting of select cross peaks of HR2 residues in the HSQC spectra, showing that MFI8 directly interacts with the HR2 domain of MFN2. Additionally, similar cross peaks shifted upon titration of the 398-418Gly peptide, indicating interaction with similar HR2 residues for both MFI8 and 398-418Gly. Notably, titration of MFI8 to 15N-labeled MFN2-HR2 S685A mutant under the same conditions showed lack of peak shifts or peak broadening indicating lack of MFI8 binding to the MFN2-HR2 S685A mutant.

To further confirm that MASM7 and MFI8 directly interact with the HR2 domain, MFN2-HR2 domain (residues 678-757) was produced and evaluated for their interaction in comparison with 367-384Gly and 398-418Gly peptides using microscale thermophoresis (MST). MASM7 and MFI8 demonstrated direct binding to the HR2 domain of MFN2 with Kds in the low micromolar range and that was comparable to the binding interactions of 367-384Gly and 398-418Gly peptides, respectively. Furthermore, MASM23 and MFI23 demonstrated direct binding to the HR2 domain of MFN2, in line with our previous results that showed that these small molecules are capable of increasing or decreasing the mitochondrial aspect ratio, respectively. In contrast, MASM19, MASM21, MASM22, MFI22, MFI25 and MFI26 did not demonstrate measurable binding to the HR2 domain of MFN2, in agreement with the inability or weak activity of these compounds to promote mitochondrial fusion or fision, respectively.

Next it was determined whether MFI8 also interacted with the HR2 domain of MFN2 in cells. MASM7 markedly increased mitochondrial aspect ratio in MFN1/MFN2 DKO MEFs when reconstituted with WT MFN2, but not when reconstituted with D725A/L727A or L727A MFN2 mutants, underscoring that MASM7 specifically targets the HR2 domain of MFN2 in cells. Likewise, MFI8 significantly decreased mitochondrial aspect ratio in MFN1/MFN2 DKO MEFs when reconstituted with WT MFN2, but not when reconstituted with S685A or L692A MFN2 mutants, indicating that MFI8 interacts specifically with the HR2 domain of MFN2 in cells. Collectively, MST, NMR and mitochondrial morphology data supported that MASM7 and MFI8 specifically interact with the HR2 domain as predicted from the pharmacophore models.

MASM7 and MFI8 modulates MFN conformation and complexes. Next, it was investigated whether MASM7 and MFI8 can modulate MFN2 oligomerization in its native membrane environment. Thus, isolated mitochondria from MEFs were treated with MASM7 or MFI8 and the capacity of MFN2 to form oligomers was monitored using blue native gel electrophoresis (BN-PAGE). MFN2 migrated as a dimer in the absence of GTP, while incubation with GTP promoted higher order oligomers ˜450 kD. Indeed, treatment of MASM7 in isolated mitochondria increased the ratio of higher order oligomers ˜450 kD to dimers upon GTP binding, whereas MFI8 reduced the ratio.

Next, cellular extract thermal shift assay (CETSA), an established assay for small molecule-protein target engagement in intact cells was used. Interestingly, MASM7 induced stabilization of MFN2 as it increased its melting point (Tₘ) by 2.3° C. (Veh: Tm=42.9±0.8° C. vs MASM7: Tm=45.2±0.5° C.) (. Furthermore, the melting curves of MFN2 were obtained with or without MASM7 from MFN2 KO MEFs reconstituted with WT and D725A/L727A MFN2. Notably, MASM7 induced stabilization of WT MFN2 (Veh: Tm=42.2±0.6° C. vs MASM7: Tm=45.3±2.3° C.), but not of D725A/L727A MFN2 (Veh: Tm=44.1±1.7° C. vs MASM7: Tm=43.3±1.7° C.).

In contrast, MFI8 alone had no effect on the Tₘ of MFN2 (Veh: Tm=42.9±0.8° C. vs MFI8: Tm=43.7±1.1° C.). Given that MFI8 binds in vitro and in cells on the HR2 domain of MFN2, reduces the ratio of higher order MFN2 oligomers to dimers and inhibits mitochondrial fusion in a MFN dependent manner, it was speculated that MFI8 engages better with the pro-tethering conformation of MFN2 in which the HR2 domain is exposed in the cytoplasm. To test this hypothesis cells were co-treated with MFI8 and MASM7, where the latter compound was found promote the pro-tethering conformation and increase the Tm of MFN2. Strikingly, MFI8 revoked MASM7-induced MFN2 stabilization (Veh: Tm=42.9±0.8° C. vs MFI8+MASM7: Tm=44.05±2.6° C.). This result is consistent with previous experiments showing that MFI8 can still promote mitochondrial fragmentation even when co-treated with MASM7. Besides target engagement, MASM7-induced MFN2 stabilization can also be attributed to the increased complexation of MFN2 with other proteins, presumably MFN1 and MFN2 as part of their functional oligomerization to mediate mitochondrial fusion. Thus, the effect of MFI8 treatment on MASM7-induced MFN2 stabilization indicates that MFI8 destabilizes homotypic (MFN2-MFN2) or heterotypic (MFN1-MFN2) complex formation.

It is noteworthy that neither MASM7 nor MFI8 altered MFN1 and MFN2 gene expression and their corresponding protein levels. Furthermore, neither MASM7 nor MFI8 altered Tomm20 protein levels, mitotracker green intensity and mitochondrial to nuclear DNA ratio, suggesting that none of the compounds altered mitochondrial biomass. In line with this result, no alteration in the gene expression levels of mitochondrial biogenesis markers was detected upon MASM7 or MFI8 treatment.

Modulation of mitofusins activity alters mitochondrial respiration and functionality. Previous reports showed that knockdown of MFN2 or knockout of both MFNs reduced respiration, suggesting that mitofusins positively regulate mitochondrial respiration. Given that MASM7 positively and MFI8 negatively regulate MFNs' fusogenic activity, it was examined whether they can affect mitochondrial respiration. Strikingly, MASM7 increased basal and maximal respiration and mitochondrial ATP production in WT but not in MFN1/MFN2 DKO MEFs. On the other hand, MFI8 reduced respiration and mitochondrial ATP production in WT but not in MFN1/MFN2 DKO MEFs. Importantly, MFI8 did not alter the ratio of state III/state II respiration of isolated mitochondria, indicating that MFI8 does not impact directly the electron transport chain (ETC) or is an unspecific uncoupler to isolated mitochondria but it rather reduces mitochondrial respiration by modulating mitochondrial dynamics. These results support the idea that the fusogenic activity of MFNs positively correlates with mitochondrial respiration.

To further support this idea WT MFN2 or β-Galactosidase (βGal) was reconstituted in MFN1/MFN2 DKO MEFs and the mitochondrial membrane potential was evaluated using TMRE staining as a readout. Interestingly, cells that expressed WT MFN2 possessed a higher mitochondrial membrane potential compared to cells that expressed βGal. Consistently, MASM7 concentration responsively increased mitochondrial membrane potential in WT MEFs. In line with this result, MASM7 significantly increased mitochondrial membrane potential of MFN1/MFN2 DKO MEFs when reconstituted with WT MFN2 and to a lesser extent when reconstituted with L727A MFN2. Importantly, MASM7-induced increase in mitochondrial membrane potential was revoked when cells were co-treated with myxothiazol or rotenone, indicating that the increase in the membrane potential upon MASM7 treatment is derived from an increased activity of the ETC. On the other hand, MFI8 concentration responsively decreased mitochondrial membrane potential in WT MEFs. It is noteworthy that MFI8 significantly decreased mitochondrial membrane potential of MFN1/MFN2 DKO MEFs when reconstituted with WT MFN2 but not when reconstituted with L692A MFN2. Strikingly, MFI8 reduced the gene expression of several nuclear-encoded subunits of the respiratory complexes as revealed by RNA-seq analysis, while MASM7 altered expression of selected genes of the respiratory complexes rather than inducing a consistent trend. Such result is in line with the idea that MFI8 promotes mitochondrial dysfunction and highlights that alterations in mitochondrial dynamics can affect gene transcription.

Next, it was determined whether such alterations can have an impact on the metabolites of the TCA cycle. Surprisingly, MASM7 had no significant effect in the majority of the metabolites of the TCA cycle. On the contrary, MFI8 markedly reduced several metabolites such as malate, oxaloacetate, and α-ketoglutarate. Consistently, MFI8 decreased the total NAD⁺/NADH ratio, which is consistent with reduced oxidative capacity. In contrast, MASM7 had no effect on total NAD⁺/NADH ratio. Collectively, these data indicate that MFNs act as regulators of mitochondrial functionality and that mitochondrial fusion positively correlates with mitochondrial functionality.

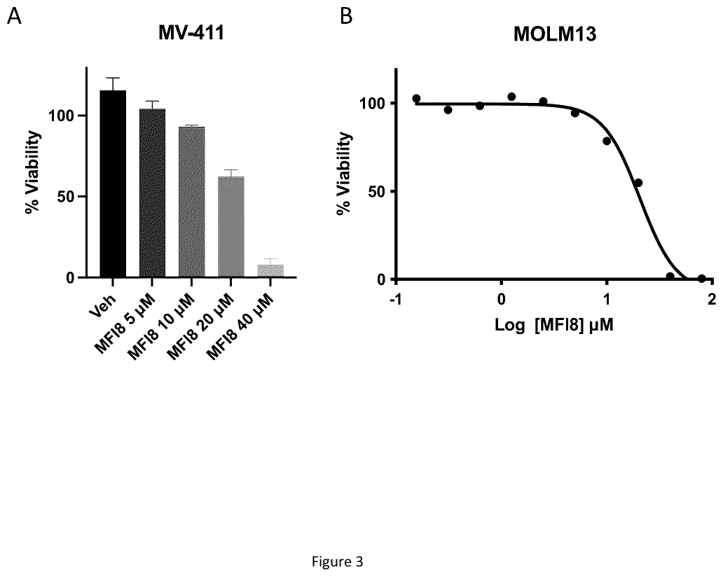

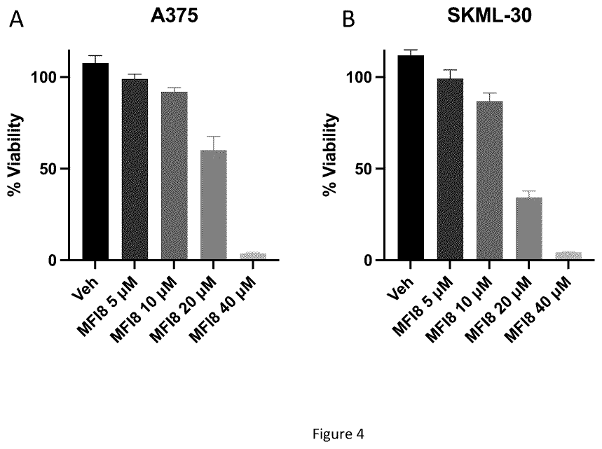

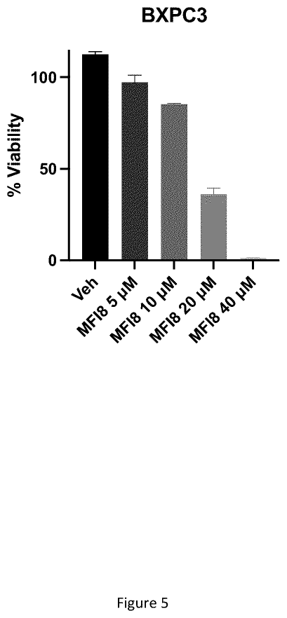

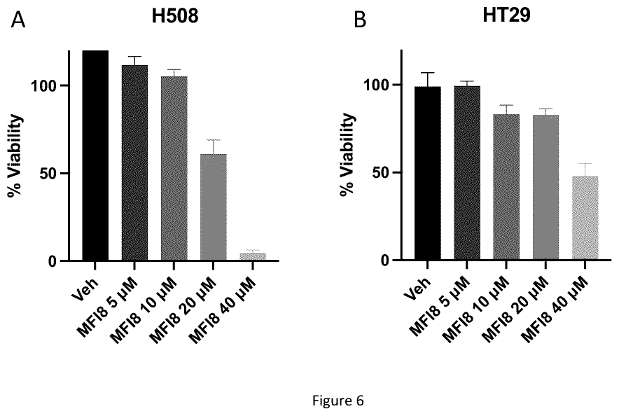

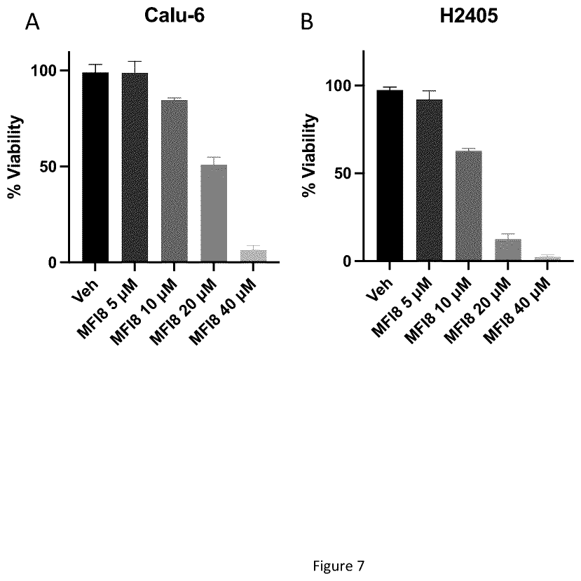

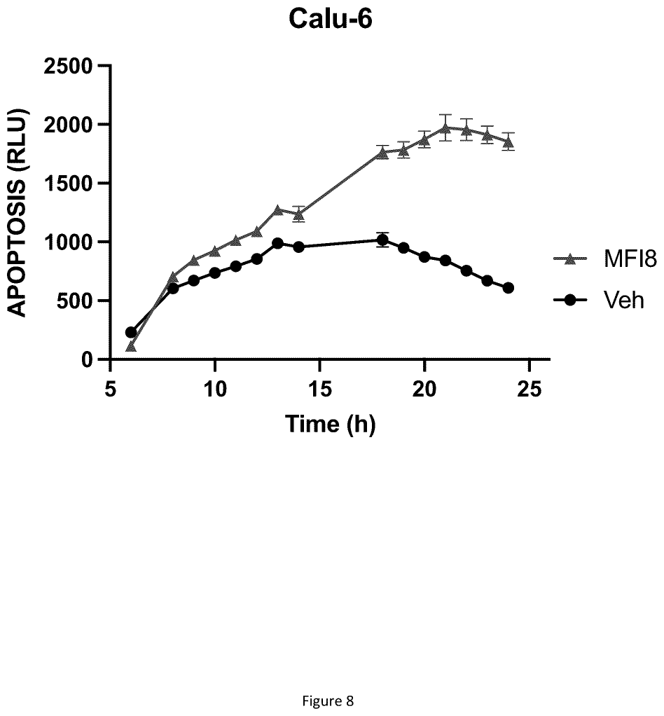

Inhibition of mitofusin activity induces minority MOMP and primes cells for cell death. Previous reports highlighted the importance of mitochondrial shape and function in the execution and sensitivity of the cells to various types of cell death, such as apoptosis. Remarkably, MFI8 concentration-responsively increased caspase-3/7 activity, a key component of apoptotic signaling, whereas MASM7 had no effect on it. Furthermore, an analogue of MFI8 that is unable to inhibit MFNs' fusogenic activity and induce aberrant mitochondrial fragmentation, MFI22, did not increase caspase-3/7 activity. Consistently, deletion of MFN1/MFN2 also impaired the capacity of MFI8 to increase caspase-3/7 activity. Taken together these data indicate that MFI8 induces caspase 3/7 activation in a MFNs dependent manner, and such phenotype is associated with mitochondrial fragmentation. Deletion of APAF-1 was detrimental for the capacity of MFI8 to increase caspase 3/7 activity, indicating that apoptosome formation is crucial for the MFI8-induced caspase 3/7 activation. Furthermore, cytosolic and mitochondrial fractions were also analyzed upon treatment with MFI8 and it was found that cytochrome c was released to the cytosol, albeit at modest levels and in a mitofusin dependent manner. Interestingly, MFI8 did not increase the percentage of dead cells upon caspase 3/7 activation. Consistently, neither MFI8 nor MASM7 decreased cellular viability over the course of 72 hours. FIGS. 3-8 further illustrates that MFI8 induces apoptosis in Calu-6 cells and various cancer cell lines.

Since cytochrome c release and caspase-3/7 activation was detected upon MFI8 treatment, it was posited that inhibition of mitochondrial fusion by MFI8 could induce mitochondrial outer membrane permeabilization (MOMP). Using an established live cell imaging assay in U2OS cells that expressed fluorescent cytosolic (CytoGFP) and mitochondrial (MitoCherry) probes, MOMP was quantified. Notably, MFI8 induced MOMP, albeit at modest levels, Accordingly, MFI8 but not MASM7 induced caspase-3/7 activation in U2OS cells. Of note, MFI8 also inhibited mitochondrial fusion in U2OS cells. Taken all together these data indicate that MFI8 induced minority MOMP and subsequent activation of caspases 3/7, albeit at sub-lethal levels. Previously, it was reported that minority MOMP induces genomic instability. Hence, the capacity of MFI8 in inducing DNA damage using yH2AX as a readout was evaluated. Interestingly, MFI8 increased yH2AX foci in WT MEFs but not in MFN1/MFN2 DKO MEFs, demonstrating that MFI8 induces DNA damage in a MFNs dependent manner. Of note, MASM7 did not induce DNA damage in any of the cell lines. Consistently, MFI8 up-regulated several genes that are involved in DNA damage response in MEFs. Moreover, MFI8 induced DNA damage in U2OS cells. Importantly, co-treatment of a pan-caspase inhibitor, Q-VD-OPh, with MFI8 abolished the capacity of the latter to induce DNA damage in U2OS.

It was further investigated if induction of minority MOMP by MFI8 can be used to enhance the capacity of another pro-apopotic agent to induce cell death. Thus, BV6, a bivalent SMAC mimetic that induces caspase-dependent cell death predominantly via XIAP inhibition was used. Notably, deletion of MFNs sensitized cells to BV6 treatment. Consistently, MFI8 potentiated the capacity of BV6 to induce cell death. The effect of MFI8 was specific to MFN1/2 inhibition as MFI22 did not sensitize cells to BV6 treatment. Importantly, deletion of MFN1/2 and APAF abolished the capacity of MFI8 to sensitize cells to BV6 treatment. Collectively, these results demonstrate that inhibition of MFNs' fusogenic activity induces minority MOMP and sub-lethal caspase-3/7 activation, which leads to DNA damage and can sensitize cells to apoptotic cell death in combination with a SMAC-mimetic. Further experiments showed that MFI8 synergizes with SMAC mimetic BV6 in THP-1 cells. More specifically, it was observed that MFI8 and SMAC mimetic BV6 synergistically induce cell death in THP-1 and U2OS cells.

It was also discovered that MFI8 synergizes with BH3 mimetic (MCL-1 inhibitor) AMG-176 in MOLM13 cells. Results showed that MFI8 sensitizes cells to caspase activation in response to BH3 mimetic (MCL-1 inhibitor) AMG-176 treatment. An increase in caspase 3/7 activation was observed in MOLM13 cells treated with increasing concentrations of AMG-176 and MFI8 (n=3, mean+SD).

These studies demonstrated direct binding of the mitofusin activator MASM7 and mitofusin inhibitor MFI8 to the recombinant HR2 domain of MFN2 and to intact MFN2 localized at mitochondria in cells. Binding and activity data come in agreement with our pharmacophore models where MASM7 and MFI8 were designed to interact with the HR2 residues based on the side chains of HR1 residues. MASM7 promotes the pro-tethering conformation of MFNs to enable mitochondrial fusion, whereas MFI8 impedes mitochondrial fusion by directly interfering with the tethering permising structure of MFNs. MASM7 and MFI8 were found to increase or decrease, respectively, the GTP-dependent MFN2 higher-order oligomers, demonstrating these small molecules can modulate the levels of pro-fusion oligomers, and therefore the extent of fusion among mitochondria. These data also support that the activity of MASM7 and MFI8 through their interactions with the HR2 domain of MFN1/2 is compatible with the proposed GTP-mediated dimerization mechanism of the GTPase domains of MFNs. The data indicated that MASM7 can activate both MFN2 and MFN1, while MFI8 can inhibit both MFN2 and MFN1. This can be attributed to the high sequence homology between MFN1 and MFN2, and the conservation of the residues that are located in the binding region of each small molecule between MFN1 and MFN2.

MFNs modulators reported here allow temporal manipulation of the fusogenic activity of MFNs in a reversible fashion. This is in contrast to other small molecules that have been reported such as the drug Leflunomide, which alters MFNs protein levels through loss of pyrimidine synthesis and are likely to affect non-fusogenic functions of MFNs. Importantly, the rational discovery of MASM7 and MFI8 enables development of novel therapeutics for disorders/syndromes where impaired mitochondrial dynamics contributes to pathogenesis. Defective MFN2 mutants have been associated with development of Charcot-Marie-Tooth disease type 2A (CMT2A) and imbalances in mitochondrial dynamics have been linked to metabolic disorders such as type II diabetes, obesity, neurodegeneration, cancer and aging. Several mutations on the GTPase, HR1 and HR2 domain of the Mfn2 have been identified from patient samples and correlated with the development of the CMT2A disease. Interestingly, M376, a residue that was used for the development of the pharmacophore model for the discovery of the MFN2 activators, has been found to be mutated in patient samples to Val. Moreover, Leu724, a residue that is located in the binding region of the MFN2 activators, has been found to be mutated in patient samples to Pro. These patient mutations highlight the importance of those residues in HR1 and HR2 domains for the function of the protein. Notably, MFN2 activators have been shown to rescue mitochondrial defects of CMT2A in preclinical models raising the opportunity for the development of novel therapeutics.

All references cited herein are incorporated by reference in their entireties.

It will be appreciated by persons skilled in the art that the invention described herein is not limited to what has been particularly shown and described. Rather, the scope of the invention is defined by the claims which follow. It should further be understood that the above description is only representative of illustrative examples of embodiments. The description has not attempted to exhaustively enumerate all possible variations. The alternate embodiments may not have been presented for a specific substituent of the compound, or a step of the method, and may result from a different combination of described substituent or step, or that other undescribed alternate embodiments may be available for a compound or method, is not to be considered a disclaimer of those alternate embodiments. It will be appreciated that many of those un-described embodiments are within the literal scope of the following claims, and others are equivalent.

1. A compound of formula I or a pharmaceutically acceptable salt thereof,
Ar¹-L-X—Ar² Formula I
wherein
Ar¹ is a 6-membered aryl or 6-membered heteroaryl, wherein the aryl or heteroaryl is substituted with one more substituents selected from the group consisting of deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl) C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂, provided that at least one of the one or more substituents is a first ortho substituent positioned ortho to L and is a hydrogen-bond donor;
Ar² is a 6-10 membered aryl or 5-10 membered heteroaryl, wherein the aryl or heteroaryl is substituted with one or more substituents selected from the group consisting of deuterium, OC₁₋₆alkyl, SC₁₋₆alkyl, CN, OH, SH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁-6alkyl, dihydroxyC₁₋₁₀alkyl, C₃₋₆cycloalkyl, C(═NC₁₋₆alkyl) C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁-6alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁-6alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂, provided that at least one of the one or more substituents is a second ortho substituent positioned ortho to X and is selected from the group consisting of OC₁₋₆alkyl, SC₁₋₆alkyl, C₁₄alkyl, CN, halogen, C₁₋₆alkylene-CN, OC₁₆alkylene-CN, haloC₁₋₆alkyl, SC₁₋₆alkylene-CN, C₂₋₆alkynyl, C₂₋₆alkenyl, and C₁₋₆alkylSO₂ (sulfone);
L is C₁₋₃alkylene optionally substituted with an oxo (═O);
X is C₁₋₃alkylene or NRⁿ;
R^{m} each is independently hydrogen or C₁₋₆alkyl or halo-C₁₋₆alkyl; and
Rⁿ is hydrogen, C₁₋₆alkyl, halo-C₁₋₆alkyl, C(O)C₁₋₆alkyl.

2. The compound of formula (I) or the pharmaceutically acceptable salt thereof of claim 1,
wherein Ar¹ is a substituted phenyl 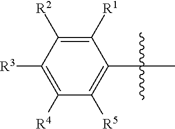
Wherein the first ortho substituent is R¹ selected from the group consisting of OH, SH, COOH, N(R^{m})₂, C(O)N(R^{m})₂, hydroxyC₁₋₆alkyl, dihydroxyC₁₋₁₀alkyl, C₃₋₆cycloalkyl, NR^{m}SO₂C₁₋₆alkyl, and S(O)OH, wherein at least one R^{m} in N(R^{m})₂, C(O)N(R^{m})₂, and NR^{m}SO₂C₁₋₆alkyl is hydrogen;
R², R³, R⁴, and R⁵ are independently selected from the group consisting of hydrogen, deuterium, OC₁₋₆alkyl, SC₁₋₆alkyl, CN, OH, SH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl) C₁₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁-6alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁-6alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂.

3. The compound of formula (I) or the pharmaceutically acceptable salt thereof of claim 1, wherein Ar¹ comprises, in addition to the first ortho substituent, one or more substituents selected from the group consisting of CN, halogen, NO₂, C(O)OR^{m}, C(O)N(R^{m})₂, C(O)C₁₋₆alkyl, haloC₁₋₆alkyl, haloC₁₋₆alkyleneO, C₁₋₆alkyl, C(O)SR^{m}, C₂₋₆alkynyl, C₂-6alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂.

4. The compound of formula (I) or the pharmaceutically acceptable salt thereof of any one of claim 1, wherein the first ortho substituent is OH.

5. The compound of formula (I) or the pharmaceutically acceptable salt thereof of claim 4, wherein R⁴ is halogen.

6. The compound of formula (I) or the pharmaceutically acceptable salt thereof of claim 5, wherein Ar² is a substituted phenyl, 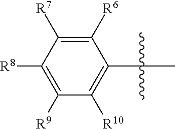
wherein the second ortho substituent is R⁶ selected from the group consisting of OC₁₋₆alkyl, SC₁₋₆alkyl, C₁₋₄alkyl, CN, halogen, C₂₋₆alkynyl, and C₂₋₆alkenyl,
R⁷, R⁸, R⁹, and R¹⁰ are independently selected from the group consisting of hydrogen, deuterium, OC₁₋₆alkyl, SC₁₋₆alkyl, CN, OH, SH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁-6alkyl, dihydroxyC₁₋₁₀alkyl, C₃₋₆cycloalkyl, C(═NC₁₋₆alkyl) C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁-6alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂.

7. The compound of formula (I) or the pharmaceutically acceptable salt thereof of claim 6, wherein R⁷ is selected from the group consisting of C₁₋₄alkyl, halogen, and haloC₁₋₆alkyl.

8. The compound of formula (I) or the pharmaceutically acceptable salt thereof claim 6, wherein R⁶ is C₁₋₄alkyl.

9. The compound of formula (I) or the pharmaceutically acceptable salt thereof of claim 6, wherein R⁶ and R⁷ are independently selected from the group consisting of OC₁₋₆alkyl, C₁₋₄alkyl, and halogen.

10. The compound of formula (I) or the pharmaceutically acceptable salt thereof of claim 9, wherein at least one of R⁸, R⁹, and R¹⁰ is a halogen.

11. The compound of formula (I) or the pharmaceutically acceptable salt thereof of claim 6, wherein L is SO₂.

12. The compound of formula (I) or the pharmaceutically acceptable salt thereof of claim 11, wherein L is methylene or CH(CH₃).

13. The compound of formula (I) or the pharmaceutically acceptable salt thereof of claim 6, wherein L is C(O).

14. The compound of formula (I) or the pharmaceutically acceptable salt thereof of claim 6, wherein X is NH.

15. The compound of formula (I) or the pharmaceutically acceptable salt thereof of claim 6, wherein X is NC(O)C₁₋₆alkyl.

16. The compound of formula (I) or the pharmaceutically acceptable salt thereof of claim 1, wherein the compound is selected from the group consisting of

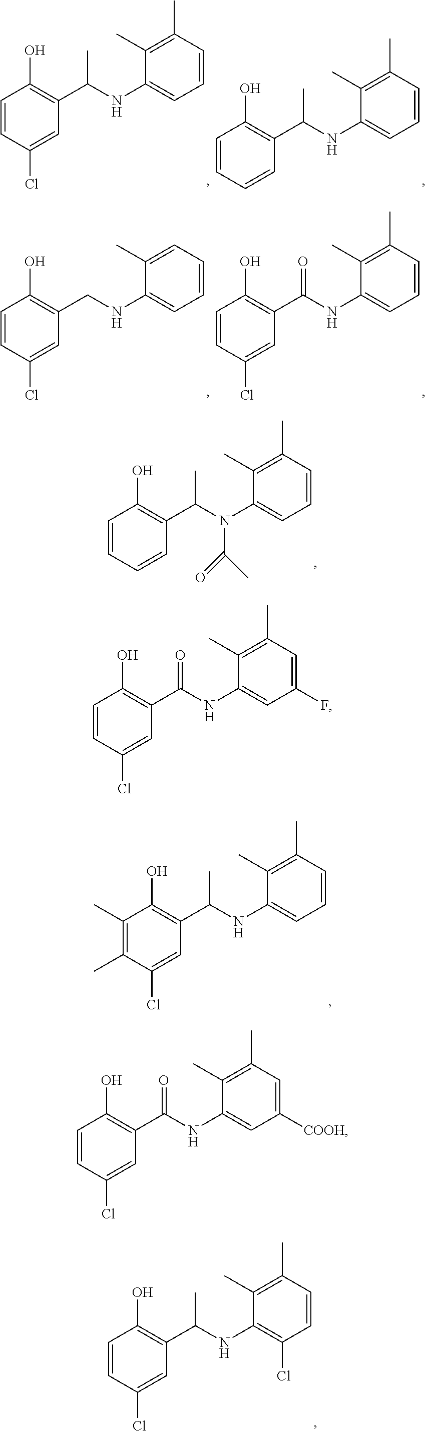

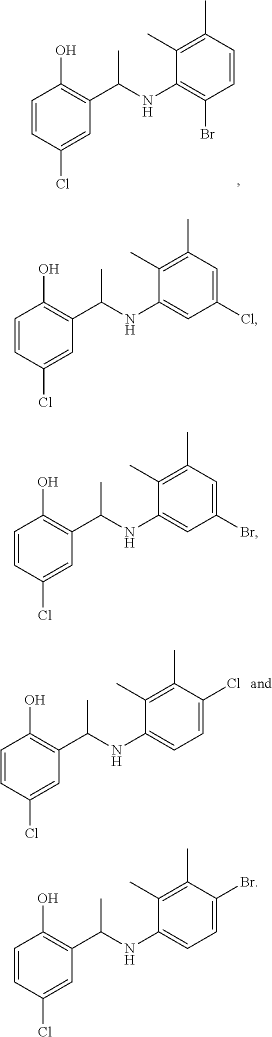 [SMILES: Cc1ccc(Br)c(NC(C)c2cc(Cl)ccc2O)c1C ; Cc1cc(Cl)cc(NC(C)c2cc(Cl)ccc2O)c1C ; Cc1cc(Br)cc(NC(C)c2cc(Cl)ccc2O)c1C ; Cc1c(Cl)ccc(NC(C)c2cc(Cl)ccc2O)c1C ; Cc1c(Br)ccc(NC(C)c2cc(Cl)ccc2O)c1C]

17. A pharmaceutical composition comprising the compound of formula (I) or the pharmaceutically acceptable salt thereof of claim 1 and a pharmaceutically acceptable carrier.

18. A method of treating a disease associated with abnormal mitochondrial fusion and/or mitochondrial fission, comprising administering to a subject in need thereof a therapeutically effective amount of the compound of formula (I) or the pharmaceutically acceptable salt thereof of claim 1, wherein the disease is selected from the group consisting of neurodegenerative diseases, metabolic disease, cardiovascular diseases, autoimmune disease, and cancer.

19. The method of claim 18, wherein the disease is selected from the group consisting of Leukemia, Acute Lymphoblastic Leukemia (ALL), Acute Myeloid Leukemia (AML), Chronic Lymphoblastic Leukemia (CLL), Chronic Myelogenous Leukemia (CML), Lymphoma, Hodgkin Lymphoma, Non-Hodgkin Lymphoma, Lung cancer, Non-Small Cell Lung Cancer, Small Cell Lung Cancer, Melanoma, Prostate cancer, Brain Tumors, Breast Cancer, Bladder Cancer, Collorectal Cancer, Head and Neck Cancer, Ovarian Cancer, Pancreatic Cancer, Hepatocellular (Liver) Cancer, Renal Cell (Kidney) Cancer, Skin Cancer, Ewing Sarcoma, Rhabdomyosarcoma, Osteosarcoma, Soft tissue sarcoma, obesity, mitochondrial disorder, diabetes, insulin resistance, sarcopenia, acute liver failure, liver fibrosis, nonalcoholic steatohepatitis, hepatosteatosis, alcoholic fatty liver, kidney fibrosis, chronic kidney disease, amyotrophic lateral sclerosis, Friedreich ataxia, spinal muscular atrophy, prion diseases, mitochondrial encephalopathy, age-related macular degeneration, glaucoma, diabetic retinopathy, retinitis pigmentosa. nonalcoholic steatohepatitis (NASH), renal failure, chronic kidney disease, Alzheimer's disease, Lewy body dementia, frontotemporal dementia, traumatic brain injury, prion diseases, Huntington's disease, Parkinson's disease, chronic traumatic encephalopathy, amyotrophic lateral sclerosis, mixed dementias, vascular dementia, and hydrocephalus.

20. A method of inhibiting mitofusin-mediated mitochondrial fusion, comprising contacting a cell with an effective amount of the compound of formula (I) or the pharmaceutically acceptable salt thereof of claim 1 to inhibit mitofusin 1 and/or mitofusin 2.


SEQUENCE LISTING (20 sequences)

SEQ ID NO: 1 - DNA, 30 bp, synthetic construct
  source 1..30: mol_type = other DNA
ctggctccaactgcgcccaccaagtccagc

SEQ ID NO: 2 - DNA, 33 bp, synthetic construct
  source 1..33: mol_type = other DNA
ccaagtccagcaggaagcgtctgggacctttgc

SEQ ID NO: 3 - DNA, 30 bp, synthetic construct
  source 1..30: mol_type = other DNA
tgggtagcaactgcgcccaccaggtgcagc

SEQ ID NO: 4 - DNA, 30 bp, synthetic construct
  source 1..30: mol_type = other DNA
aggtgcagcaagaggcgagcggcaccttcg

SEQ ID NO: 5 - DNA, 45 bp, synthetic construct
  source 1..45: mol_type = other DNA
aagaaaattgaagttctggccagcgcgcaaagcaaggcgaaactg

SEQ ID NO: 6 - DNA, 39 bp, synthetic construct
  source 1..39: mol_type = other DNA
aattgaagttctggacagcgcgcaaagcaaggcgaaact

SEQ ID NO: 7 - DNA, 20 bp, synthetic construct
  source 1..20: mol_type = other DNA
caaaatcgccctattcctca

SEQ ID NO: 8 - DNA, 20 bp, synthetic construct
  source 1..20: mol_type = other DNA
agacccagcttcgttctcct

SEQ ID NO: 9 - DNA, 20 bp, synthetic construct
  source 1..20: mol_type = other DNA
gaccgaagggtcagatgaaa

SEQ ID NO: 10 - DNA, 20 bp, synthetic construct
  source 1..20: mol_type = other DNA
tccagctctgtggtgacatc

SEQ ID NO: 11 - DNA, 20 bp, synthetic construct
  source 1..20: mol_type = other DNA
cctggatgctgatgtgtttg

SEQ ID NO: 12 - DNA, 20 bp, synthetic construct
  source 1..20: mol_type = other DNA
ccagctcatccaccagaaag

SEQ ID NO: 13 - DNA, 26 bp, synthetic construct
  source 1..26: mol_type = other DNA
tgatgtgaatgacttggatacagaca

SEQ ID NO: 14 - DNA, 26 bp, synthetic construct
  source 1..26: mol_type = other DNA
gctcattgttgtactggttggatatg

SEQ ID NO: 15 - DNA, 22 bp, synthetic construct
  source 1..22: mol_type = other DNA
gggaaagggaccagacataatc

SEQ ID NO: 16 - DNA, 22 bp, synthetic construct
  source 1..22: mol_type = other DNA
gcggaagcagatggtaagataa

SEQ ID NO: 17 - DNA, 20 bp, synthetic construct
  source 1..20: mol_type = other DNA
cctatcacccttgccatcat

SEQ ID NO: 18 - DNA, 20 bp, synthetic construct
  source 1..20: mol_type = other DNA
gaggctgttgcttgtgtgac

SEQ ID NO: 19 - DNA, 20 bp, synthetic construct
  source 1..20: mol_type = other DNA
atggaaagcctgccatcatg

SEQ ID NO: 20 - DNA, 20 bp, synthetic construct
  source 1..20: mol_type = other DNA
tccttgttgttcagcatcac